(19) United States
(12) Patent Application Publication
(10) Pub. No.: US 2022/0118057 A1
(43) Pub. Date: Apr. 21, 2022

(54) ALBUMIN NANOPARTICLES, THE MAKING METHOD, AND USES THEREOF

(71) Applicant: NOVAGENESIS THERAPEUTIX (HK) LIMITED, Hong Kong (CN)

(72) Inventors: Feng QIAN, Beijing (CN); Chun LIU, Beijing (CN)

(21) Appl. No.: 17/424,616

(22) PCT Filed: Jan. 22, 2020
(86) PCT No.: PCT/CN2020/073707

§ 371 Date: Jul. 21, 2021

(30) Foreign Application Priority Data
Jan. 22, 2019 (CN) 201910058327.7

(51) Int. Cl. A61K 38/38; A61K 9/51; A61K 9/19; A61P 35/00; A61K 31/352

(52) U.S. Cl. CPC A61K 38/385; A61K 9/5192; A61K 31/352; A61P 35/00; A61K 9/19

(57) ABSTRACT

The present disclosure provides an albumin nanoparticle comprising a core consisting of at least one diester derivative of β-lapachone and a shell consisting of at least one albumin. Further, herein provided are a preparation method and use thereof. The albumin nanoparticle has a significantly longer in vivo half-life, markedly reduced toxicity and side effects, and an observably widened therapeutic window, and thus can treat cancer associated with KRAS mutations.

### TECHNICAL FIELD

The present disclosure relates to an albumin nanoparticle, comprising a core consisting of diester derivative of β-lapachone and a shell consisting of albumin, wherein the core and the shell are connected with a non-covalent bond. The present disclosure also relates to the making methods and uses of the albumin nanoparticles, such as use of the albumin nanoparticles in treatment of Kirsten Rat Sarcoma 2 Viral Oncogene Homolog (“KRas”) associated cancers.

### BACKGROUND

KRas is a small GTPase and a member of the Ras family of oncogenes. KRas serves a molecular switch cycling between inactive (GDP-bound) and active (GTP-bound) states to transduce upstream cellular signals received from multiple tyrosine kinases to downstream effectors to regulate a wide variety of processes, including, for example, cellular proliferation (see, e.g., Alamgeer et al., Current Opin Pharmcol. 13:394-401 (2013)).

The role of activated KRas in malignancy was observed over thirty years ago (see, e.g., Santos et al., Science 223:661-664 (1984)). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (see, e.g., Samatar et al.,Nat. Rev. Drug Disc. 13(12): 928-942 (2014)). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (see, e.g., Dogan et al., Clin Cancer Res. 18(22):6169-6177 (2012)). The well-known role of KRAS in malignancy and the discovery of these frequent mutations in KRas in various tumor types made KRas a highly attractable target of the pharmaceutical industry for cancer therapy.

Pancreatic cancer, currently the 4^{th} most common cause of cancer death in the US (see, Siegel et al., CA Can. J Clin., 64(1):9-29 (2014)) is the most lethal of all cancers with an overall 5-year survival under 5% (see, Wolfgang et al., CA: A Can. J Clin., 63(5):318-48 (2013)). Each year, the number of pancreatic cancers diagnosed nearly equals the number of deaths for the disease. This dismal outcome has not changed over the past three decades, despite the efforts to improve therapy. Id. Of grave concern, pancreatic cancer is on the rise. In the US, pancreatic cancer is anticipated to become the second most common cause for cancer death by 2030 (see, Rahib et al., Can. Res., 74(14):2913-2921 2014)). In other parts of the world, pancreatic cancer is only one of the cancers with a worsening forecast, whereas the incidence and mortality of other common cancers (e.g., breast, colon, and prostate) are trending downward (see, Malvezzi et al., Annals of Onco.: Offi. J Eur. Soc. Med. Onco./ESMO, 2014). Pancreatic ductal adenocarcinoma (“PDAC”) accounts for majority form (estimated about 80-85%) of pancreatic cancer. And PDAC is also one of the most lethal solid tumors with a median survival time of only 6 months and an overall 5-year survival rate lower than 5% (see, Seufferlein et al., Transl. Gastroenterol. Heptal. 4:21 (2019)).

Effective treatment for pancreatic cancer will likely require use of agents that can cause tumor-specific cell death, independent of oncogenic driver mutations such as Kras and p53, or apoptotic processes (e.g., caspase-independent). KRas-mutant-driven NAD(P)H:quinone oxidoreductase 1 (NQO1) is over-expressed in pancreatic tumor versus associated normal tissue, while catalase expression is lowered compared to levels in associated normal pancreas tissue.

NAD(P)H:quinone oxidoreductase 1 (NQO1 a.k.a. DT-diaphorase) expression is an exploitable, tumor-specific target for pancreatic cancer therapies. Elevated NQO1 expression is noted in many early forms of cancers, such as in pancreatic intraepithelial neoplasia (see, DeNicola et al., Nature 475:106-109 (2011)), prostatic intraepithelial neoplasia (see, e.g., Dong et al., Cancer Res. 70:8088-8096 (2010)), and breast ductal carcinoma in situ (see, e.g. Marin et al., Br J Cancer 76:923-929 (1997)) and further increase in this enzyme occurs as these cancers progress (see, Madajewski et al., Mol Cancer Res. 14:14-25 (2016)). NQO1 bioactivatable drugs are a unique class of rare quinones that include, for example, β-Lapachone and deoxynyboquinones.

β-Lapachone (herein referred as “β-lap”) has a structure of formula (β-lap)

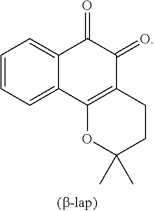 [SMILES: CC1(C)CCC2=C(O1)c1ccccc1C(=O)C2=O]

It is a natural product isolated from the Lapacho tree in the rainforest of South America. It can kill a broad spectrum of cancer cells in an NQO1-dependent manner (see, Bey et al., Proc. Natl. Acad. Sci. U.S.A. 104:11832-11837 (2007)). In cancer cells overexpressing NQO1, β-lap undergoes a futile redox cycle, resulting in a rapid and massive production of reactive oxygen species (see, Reinicke et al., Clin. Cancer Res. 11:3055-3064 (2005)). Cell death occurs specifically in cancer cells overexpressing NQO1, while normal cells and tissue with low endogenous levels of the enzyme are spared. NQO1 metabolizes β-lap into unstable hydroquinone, and then oxidizes it to a superoxide, such that poly (ADP-ribose) polymerase-1 (PARP1) is activated excessively and the programmed death of tumor cells is initiated. Whileβ-lap is a promising agent from a mechanistic standpoint, its clinical use is hampered by low water solubility, poor pharmacokinetics and methemoglobinemia.

The use of hydroxypropyl β-cyclodextrin (HPβCD) to formulate 62 -lap increased the drug's solubility in water significantly (see, Nasongkla et al., Pharm. Res. 20:1626-1633 (2003)). However, rapid drug clearance from the blood, hemolysis due to the HPβCD carrier cyclodextrin, and β-lap-induced methemoglobinemia (see, Blanco et al., Cancer Res. 70:3896-3904 (2010)) were noted, limiting its success as a therapeutic agent in clinical trials. Then development of polymeric micelles for the delivery of β-lap was reported, e.g., micelles composed of poly(ethylene glycol)-β-poly(D,L-lactic acid) (PEG-β-PLA). Id. However, such micellar formulation is limited due to its low drug loading density and efficiency, resulting from the fast crystallization of β-lap.

A prodrug strategy to improve the formulation properties of β-lap, by investigating the use of carbonic ester prodrugs of β-lap, has been developed. It was reported that diester prodrugs of β-lap have greatly improved drug loading density and efficiency in PEG-β-PLA micelles, and both dC3 and dC6 prodrugs (see the structures below) achieved advantageous results. In particular, dC3 prodrug micelles showed excellent antitumor efficacy in treating orthotopic NSCLC tumors that overexpress NQO1. See, e.g. Ma et al., Adv. Healthc. Mat. 3(8):1210-1216 (2014); Ma et al., J Control Release 200:201-211 (2015).

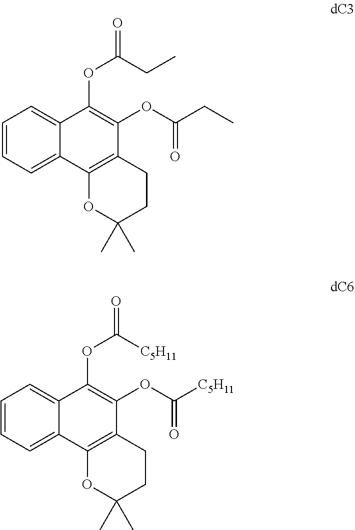

However, such prodrug micelle formulation bears a narrow therapeutic window. Thus, there remains a need to develop new prodrug formulations that demonstrate sufficient efficacy, stability and/or safety for treating KRas associated cancers, for example, pancreatic cancer, especially PDAC.

### BRIEF SUMMARY

In one aspect, the present disclosure provides an albumin nanoparticle comprising:

a core consisting of at least one diester derivative of β-lapachone having a formula of (I):

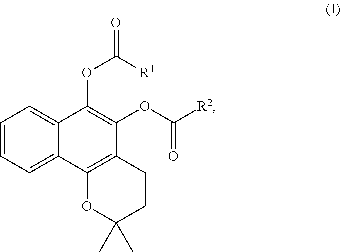

wherein
R¹ and R² are each independently selected from C₁-C₁₀ alkyl, C₂-C₆ alkenyl, C₂-C₆ alkynyl, C₃-C₈ cycloalkyl, C₆-C₁₀ aryl, C₃-C₈ heterocycloalkyl, C₈-C₁₀ heteroaryl, each of which is unsubstituted or substituted with 1 or 2 R′;
or R¹ and R² are each independently selected from a bond, —O—, —S—, —NH—, and C₁-C₄ alkylene to form a 6-10 membered ring, wherein the ring is unsubstituted or substituted with 1 or 2 R′;
further wherein each R′ is independently selected from halogen, hydroxyl, C₁-C₃ alkyl, C₁-C₃ alkoxy, C₁-C₃ haloalkyl, and —CN;
and a shell consisting of at least one albumin,
wherein the core and the shell are connected with a non-covalent bond.

In the second aspect, the present disclosure provides a process of making the albumin nanoparticle disclosed herein, comprising:
mixing the at least one diester derivative of β-lapachone with the at least one albumin to obtain a mixture;
homogenizing the mixture to obtain a homogenized mixture; and
lyophilizing the homogenized mixture.

In the third aspect, the present disclosure provides an albumin nanoparticle, made by a process selected from de-solvation, self-assembly, emulsification, double emulsification, thermo gel, spray drying, nanoparticle albumin-bound (nab) technology, and pH agglomeration.

In the fourth aspect, the present disclosure provides a pharmaceutical composition, comprising the albumin nanoparticle disclosed herein and at least one pharmaceutically acceptable carrier.

In the fifth aspect, the present disclosure provides a method of treating cancer by administering a patient in need thereof a therapeutically effective amount of the albumin nanoparticle disclosed herein.

### BRIEF DESCRIPTIONS OF THE DRAWINGS

FIGS. 1A to 1G show the results of release of dC₃ and β-lap from bovine serum albumin nanoparticles (BSA-NP) according to an embodiment of the present disclosure and polymer micelles,
wherein:

FIG. 1A shows the conversion of the prodrug dC₃ to β-lap in the presence of esterase;

FIG. 1B shows the particle size distribution of dC₃ micelles and BSA-NP, which is characterized by DLS, and shows that BSA-NPs with the original particle size of about 110 nm dissociated into that with a particle size of 5-10 nm after dilution 1000 times;

FIG. 1C shows the morphology and crystallization of two formulations, which are respectively characterized by TEM (scale bar: 200 nm) and POM (scale bar: 200 μm), showing that dC₃ crystals were precipitated from the micelles and could be observed by the POM after the micelle solution was diluted 500 times, whereas no dC₃ crystals were precipitated in the BSA-NPs solution after the BSA-NPs solution was diluted 1000 times;

FIG. 1D shows the kinetics of in vitro release of dC₃ (absence of PLE) and β-lap (presence of PLE) from micelles and BSA-NPs (**P<0.01 and *P<0.05);

FIG. 1E shows the in vitro conversion of β-lap from micelles and BSA-NPs (presence of PLE) in an albumin solution with the same albumin concentration in plasma concentration;

FIGS. 1F and 1G show the dC₃-β-lap conversion in micelles and BSA-NPs in the presence of different concentrations of BSA (at different molar ratios shown in the figures), and in the absence (1F) or presence (1G) of PEG-b-PLA at a concentration for micelle preparation, wherein the concentration of dC₃ was maintained at 25 μg/mL.

FIGS. 2A to 2D show the results of BSA-NPs according to an embodiment of the present disclosure and micelles in a systemic circulation,
wherein:

FIG. 2A shows that when PLE was 1 U/mL, compared with micelles and clinically-tested β-lap-CD preparations (ARQ 501), BSA-NPs produced much less methemoglobin within 45 minutes;

FIG. 2B shows the overall tolerance and survival rate of healthy BALB/c nude mice after intravenous injection of β-lap-CD, dC₃ micelles and BSA-NPs (via a tail vein injection, once every 3 days for a total of 4 times), wherein it was determined that the β-lap-CD, dC₃ micelles and dC₃ BSA-NP respectively had a MTD of 20, 50 and 100 mg/kg (β-lap equivalent);

FIGS. 2C and 2D show the change of dC₃ and β-lap concentrations in plasma of rats after the administration of dC₃ micelles and BSA-NPs at a dose of 50 mg/kg (β-lap equivalent, n=3).

FIGS. 3A and 3B show the KRAS mutation level and NQO1 mRNA level in four cell lines according to an embodiment of the present disclosure,
wherein:

FIG. 3A shows the expression level of NQO1 protein measured by western blot method, and

FIG. 3B shows the mRNA level measured by qPCR method, wherein normal mouse pancreas (n=3) was used as a negative control.

FIGS. 4A to 4F show the results of KRAS mutations according to an embodiment of the present disclosure, causing PDAC to be sensitive to albumin-bound dC₃, and KRAS-associated macropinocytosis affecting the cytotoxicity of different preparations,
wherein:

FIG. 4A shows toxic effects of different preparations: β-lap, β-lap with DIC (dicoumarol, an NQO1 inhibitor, 50 μM), dC₃, dC₃ micelles and dC₃ BSA-NP, in MIA PACA-2 (transfected with a control vector or pCDH-KRas-shRNA), and BxPC-3 (transfected with a control vector or pCDH-KRas-G12V) cells;

FIG. 4B shows IC 50 values of different treatments in A (**P<0.01 and *P<0.05; Errors represent the mean±variance of n=3 independent experiments);

FIG. 4C shows comparison of uptake of 6-TAMRA-BSA (as indicated by arrow 2 in the figure) by cancer cells pretreated with or without 75 μM EIPA (a known macropinocytosis inhibitor), wherein DAPI staining (as indicated by arrow 1 in the figure) was used to identify cell nuclei (scale bar: 10 μm);

FIG. 4D shows the quantification of the comparison in FIG. 4C by flow cytometry (***P<0.001);

FIGS. 4E and 4F show that cells were treated with different dC₃ preparations (2 μM β-lap equivalent) for 2 hours (before this, pre-treatment was performed with and without 75 μM EIPA half an hour in advance, respectively), wherein the concentrations of two drugs were measured by LC-MS/MS and were all expressed as β-lap in consideration of the fact that dC₃ can be converted into β-lap by enzymes in the cells.

FIGS. 5A to 5E illustrate that KRAS^{G12D} PDAC cell SW1990 according to an embodiment of the present disclosure and its gemcitabine-resistant cell line SW1990/GEM had an KRAS-associated enhanced macropinocytosis in vitro and an anti-tumor effect in vivo,
wherein:

FIG. 5A shows the cytotoxic effects on the two cell lines in the presence of β-lap, β-lap with DIC (dicoumarol, a NQO1 inhibitor, 50 μM), dC₃, dC₃ micelles and dC₃ BSA-NP;

FIG. 5B shows IC50 values for different treatments in FIG. 5A;

FIG. 5C shows comparison of uptake of FITC-BSA (as indicated by arrow 1 in the figure) by cancer cells pretreated with or without 75 μM EIPA, wherein DAPI staining (as indicated by arrow 2 in the figure) was used to identify cell nuclei (scale bar: 10 μm);

FIG. 5D shows the quantification of the comparison in figure C by flow cytometry (**P<0.01);

FIG. 5E shows the in vivo anti-tumor comparison of different β-lap treatments (once every three days for a total of 4 times) in SW1990/GEM cell-derived subcutaneous xenograft tumors in BALB/c nude mice (saline, dC₃ micelles (50 mg/kg), dC₃ BSA-NPs (50 mg/kg) and dC₃ BSA-NPs (100 mg/kg, * P<0.05)).

FIGS. 6A to 6E show experimental results of KRAS mutations according to an embodiment of the present disclosure causing the PDAC xenograft subcutaneously implanted into the BALB/c nude mice to be sensitive to albumin-bound dC₃,
wherein:

In subcutaneous xenograft tumors of BALB/c nude mice, which were derived from MIA PaCa-2 cells transfected with control vectors (6A and 6B) or pCDH-KRas-shRNA (6C and 6D), in vivo anti-tumor effects of treatments with different β-lap preparations (once every 3 days for a total of 4 times) represented as saline, β-lap-CD (20 mg/kg), dC₃ micelles (50 mg/kg), dC₃ BSA-NPs (50 mg/kg) and dC₃ BSA-NPs (100 mg/kg, ***P<0.001) in the figure were compared. and after the tumor had reached a certain level, the mice were sacrificed and the tumors were removed and weighed (****P<0.0001);

FIG. 6E shows that mice with different cell xenograft tumors were intravenously injected with dC₃ micelles and BSA-NPs at a dose of 50 mg/kg for 2 h; pre-treatment was performed with and without injection of 5 μg EIPA in the tumor half an hour in advance, and the drug concentrations in the tumor were measured by LC-MS/MS and all expressed as β-lap (*P<0.05).

FIGS. 7A to 7E show experimental results of KRAS mutations according to an embodiment of the present disclosure causing the PDAC xenograft subcutaneously implanted into the BALB/c nude mice to be sensitive to albumin-bound dC₃,
wherein:

In subcutaneous xenograft tumors of BALB/c nude mice, which were derived from BxPC-3 cells transfected with control vectors (A and B) or pCDH-KRas-G12V (C and D), in vivo anti-tumor effects of treatments with different β-lap preparations (once every 3 days for a total of 4 times) represented as saline, β-lap-CD (20 mg/kg), dC₃ micelles (50 mg/kg), dC₃ BSA-NPs (50 mg/kg) and dC₃ BSA-NPs (100 mg/kg, ***P<0.001) in the figure were compared, and after the tumor had reached a certain level, the mice were sacrificed and the tumors were removed and weighed (****P<0.0001);

FIG. 7E shows that mice with different cell xenograft tumors were intravenously injected with dC3 micelles and BSA-NPs at a dose of 50 mg/kg for 2 h; pre-treatment was performed with and without injection of 5 μg EIPA in the tumor half an hour in advance, and the drug concentrations in the tumor were measured by LC-MS/MS and all expressed as β-lap (*P<0.05).

FIGS. 8A and 8B show experimental results of anti-PDAC efficacy of albumin-bound preparations (BSA-NPs) according to an embodiment of the present disclosure compared with two early preparations (ARQ761/β-lap-CD and dC₃ micelles),
wherein:
Saline, β-lap-CD (20 mg/kg,), dC₃ micelles (50 mg/kg), and dC₃ BSA-NPs (50 mg/kg and 100 mg/kg, **P<0.01) were administered (once every 3 days for a total of 4 times) to orthotopic MIA PaCa-2 (8A) and BxPC-3 (8B) PDAC xenografts of female BALB/c mice (n=10), and Kaplan-Meier survival curves were measured.

FIGS. 9A to 9C show the results of anti-tumor effects of albumin-dC₃ according to an embodiment of the present disclosure and HPβCD-β-lap on orthotopic A549 tumors in female NOD-SCID mice,
wherein:

FIG. 9A shows BLIs of the anesthetized mice on days 20 and 35 after treatment with HPβCD (carrier only), HPβCD- β-lap (22 mg/kg) and albumin-dC₃ (22, 50 and 70 mg/kg) (day 0 is designated as the day after injection of A549 cancer cells);

FIG. 9B shows quantification of luciferase levels in mice treated with albumin-dC₃ and HPβCD-β-lap as a function of time;

FIG. 9C shows the Kaplan-Meier survival curve of female NOD-SCID mice (n=4) with orthotopic A549 NSCLC xenograft tumors after administration of HPβCD (carrier only), HPβCD- β-lap (22 mg/kg) or albumin-dC₃ (22, 50 and 70 mg/kg) (*p≤0.05; ** p≤p0.01; ***p0.001).

### DETAILED DESCRIPTION

Tumor-selectivity remains a challenge for efficacious chemotherapeutic strategies against cancer. Although the recent development of β-lapachone to specifically exploit elevated levels of NAD(P)H:quinone oxidoreductase 1 (NQO1) in most solid tumors has represented a novel chemotherapeutic approach, additional therapies that kill by various mechanisms such as programmed necrosis at increased potency are needed.

### Definitions

The following terms, unless otherwise indicated, shall be understood to have the following meanings:

As used herein, including the claims, the singular forms of words, such as “a,” “an,” and “the,” include their corresponding plural references unless the context clearly dictates otherwise. The terms “comprising”, “including”, “containing”, “having” etc. shall be read expansively or open-ended and without limitation.

The term “about” when used before a numerical designation, e.g., temperature, time, amount, concentration, and such others, including a range, indicates approximations, which may vary, for example, by (+) or (−) 10%, 5%,1%, or any subrange or subvalue there between, depending on the nature of the parameter and measurement. In some embodiments, the term “about” when used with regard to a dose amount means that the dose may vary by +/−10%.

Unless otherwise indicated, the term “at least” preceding a series of elements is to be understood to refer to every element in the series. The terms “at least one” and “at least one of” include, for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

β-lapachone (β-lap) has a structure of formula (β-lap):

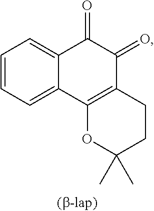 [SMILES: CC1(C)CCC2=C(O1)c1ccccc1C(=O)C2=O]

and is a natural product isolated from the Lapacho tree in the rainforest of South America. It can kill a broad spectrum of cancer cells in an NQO1-dependent manner (see, Bey et al., Proc. Natl. Acad. Sci. U.S.A. 104:11832-11837 (2007)). In cancer cells overexpressing NQO1, β-lap undergoes a futile redox cycle, resulting in a rapid and massive production of reactive oxygen species (see, Reinicke et al., Clin. Cancer Res. 11:3055-3064 (2005)).

A dash (“—”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a saturated straight or branched hydrocarbon, such as a straight or branched group of 1-8 carbon atoms, referred to herein as CC₁₋₈ alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkenyl” as used herein refers to an unsaturated straight or branched hydrocarbon having at least one carbon-carbon double bond, such as a straight or branched group of 2-6 carbon atoms, referred to herein as (C₂₋₈alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkynyl” as used herein refers to an unsaturated straight or branched hydrocarbon having at least one carbon-carbon triple bond, such as a straight or branched group of 2-6 carbon atoms, referred to herein as (C₂₋₆alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “aryl” as used herein refers to a mono-, bi-, or other multi-carbocyclic, aromatic ring system with 5 to 14 ring atoms. The aryl group can optionally be fused to one or more rings selected from aryls, cycloalkyls, heteroaryls, and heterocyclyls. The aryl groups of this present disclosure can be substituted with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to, a monocyclic aromatic ring system, wherein the ring comprises 6 carbon atoms, referred to herein as “C₆-aryl.”

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to at least one of other cycloalkyl (saturated or partially unsaturated), aryl, and heterocyclyl groups, to form, for example, a bicycle, or a tetracycle, etc. The term “cycloalkyl” also includes bridged and spiro-fused cyclic structures, which may or may not contain a heteroatom.

The term “halo” or “halogen” as used herein refers to —F, —Cl, —Br, and/or —I.

“Haloalkyl” means an alkyl group substituted with at least one halogen. Examples of haloalkyl groups include, but are not limited to, trifluoromethyl, difluoromethyl, pentafluoroethyl, trichloromethyl, etc.

The term “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with at least one substituent selected, for example, from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Heterocycles can also be selected from bicyclic, tricyclic, and tetracyclic groups, in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

“Albumin” is a globular serum protein with an approximate molecular weight of 65 kDa. The albumin disclosed herein may be selected from serum-based albumin, e.g. human serum albumin and bovine serum albumin, bioengineered recombinant albumin, e.g., ovalbumin cross-linked by at least one entity chosen from an aliphatic dialdehyde, e.g. glutaraldehyde, glyoxyl, dimethylglyoxal or ketones, e.g. 2,3-butane dione, esters, e.g. ethylene glycol bissuccin-imidyl-succinate, acid chlorides, e.g. terephthalic acid dichloride, and diisocyanates, e.g. toluene diisocyanate, or by at least one of di-, tri- and tetravalent metallic cations or by heat (90-170° C., 10-60 min), and analogs thereof. See, Tomlinson et al, Monolithic albumin particles as drug carriers. In: L. Illum, J. G. McVie and E. Tomlinson (eds.), Polymers in Controlled Drug Delivery, Wright, Bristol, 1987, pp. 25-48.

In some embodiments, the albumin is selected from serum-derived albumin, bioengineered recombinant albumin, and analogs thereof. In a further embodiment, the serum-derived albumin is selected from human serum albumin and bovine serum albumin.

The amount of albumin included in the pharmaceutical composition of the present disclosure can vary depending on, for example, the pharmaceutically active agent, other excipients, and the route and site of intended administration. Desirably, the amount of albumin included in the composition disclosed herein is an amount effective to reduce one or more side effects of the pharmaceutically active agent due to the administration of the inventive pharmaceutical composition to a human.

The term “nanoparticle” as used herein refers to particles having at least one dimension, which is usually less than 5 microns. In some embodiments, such as for intravenous administration, the nanoparticle is less than 1 micron. For direct administration, the nanoparticle can be bigger than that for intravenous administration. Even bigger particles are expressly contemplated by the present disclosure. The term “nanoparticle” may also encompass discrete multimers of smaller unit nanoparticles.

Non-covalent bond is a type of chemical bond that typically bonds between macromolecules. It does not involve sharing a pair of electrons, but rather involves more dispersed variations of electromagnetic interactions between molecules or within a molecule. Non-covalent bond can be selected, for example, from electrostatic interactions, such as ionic interaction, hydrogen bond, and halogen bond; van der Waals force, such as dipole-dipole, dipole-induced dipole and London dispersion forces; π-effects, such as π-π interaction, cation-π and anion-π interaction, and polar-π; and hydrophobic effects. In some embodiments, the non-covalent bond is hydrogen bond.

In some embodiments, the albumin nanoparticle disclosed herein has a mean particle size ranging from about 50 to about 500 nm. In a further embodiment, the mean particle size is about 110 nm.

In some embodiments, the albumin nanoparticle disclosed herein has a Poly Dispersity Index (PDI) ranging from about 0.01 to about 0.50, such as from about 0.10 to about 0.40, further such as from about 0.10 to about 0.28. In a further embodiment, the PDI is about 0.21.

In some embodiments, a molar ratio of the at least one diester derivative of β-lapachone and the at least one albumin ranging from about 50:1 to about 1:1, such as about 40:1 to about 1:1. In a further embodiment, the molar ratio of the at least one diester derivative of β-lapachone and the at least one albumin is about 20:1.

Methods of making nanoparticle compositions are known in the art. For example, nanoparticles containing taxanes (such as paclitaxel) and carrier protein (such as albumin) can be prepared under conditions of high shear forces (e.g., sonication, high pressure homogenization, or the like). See, e.g., U.S. Pat. Nos. 5,916,596; 6,506,405; and 6,537,579 and also in U.S. Patent Publication No. 2005/0004002A1. The preparation methods of albumin-based nanomedicine have also been known in the art. Literature reviews have summarized the methods, which are selected, for example, from de-solvation, self-assembly, emulsification, double emulsification, thermo gel, spray drying, nanoparticle albumin-bound (nab) technology, and pH agglomeration. See, e.g., Liu et al., Chem Soc Rev. 45:1432-56 (2016); Elzoghby et al., J Control Release. 157:168-82 (2012); and Lee et al., J Pharm Invest. 46:305-15 (2016).

In some embodiments, the present disclosure provides a process of making the albumin nanoparticle, comprising: mixing the at least one diester derivative of β-lapachone with the at least one albumin to obtain a mixture; homogenizing the mixture to obtain a homogenized mixture; and lyophilizing the homogenized mixture.

The terms “lyophilize,” “lyophilization” and the like as used herein refer to a process by which the material (e.g., nanoparticles) to be dried is first frozen and then the ice or frozen solvent is removed by sublimation in a vacuum environment. An excipient is optionally included in pre-lyophilized formulations to enhance stability of the lyophilized product upon storage.

In a further embodiment, at least one dispersing agent may be used to suspend or dissolve albumin. The dispersing agent that can be used in the present disclosure includes any liquid that is capable of suspending or dissolving biologic, but does not chemically react with either the polymer that is used to produce the shell, or the biologic itself. Examples of the dispersing agent include vegetable oils, (e.g., soybean oil, mineral oil, corn oil, rapeseed oil, coconut oil, olive oil, safflower oil, cotton seed oil, and the like), aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 1-30 carbon atoms (e.g., octanol, and the like), aliphatic or aromatic esters having 2-30 carbon atoms (e.g., ethyl capyrlate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally with at least one halogen substituent, e.g., CH₃Cl, CH₂Cl₂, CHCl₃, CH₂Cl—CH₂Cl, and the like), ketones having 3-30 carbon atoms (e.g., acetone, methyl ethyl ketone, and the like), polyalkylene glycols (e.g., polyethylene glycol, and the like), or combinations of any two or more thereof.

Examples of the combinations of dispersing agents include volatile liquids such as acetone, dichloromethane, chloroform, ethyl acetate, isopropyl acetate, benzene, methanol, ethanol, isopropanol, acetonitrile, and the like (i.e., solvents that have a high degree of solubility for the pharmacologically active agent, and are soluble in the other dispersing agent employed), along with a less volatile dispersing agent. When added to the other dispersing agent, these volatile additives help to drive the solubility of the pharmacologically active agent into the dispersing agent. This is desirable since this operation is usually time consuming. Following dissolution, the volatile component may be removed by, for example, evaporation (optionally under vacuum).

In some embodiments, the diester derivative of β-lapachone has a concentration ranging from about 50 to about 200 mg/mL after the dispersing. In some embodiments, albumin is in an aqueous solution having a concentration ranging from about 2 to about 50 mg/mL after the dispersing.

In some embodiments, the process further comprises adding the at least one diester derivative of β-lapachone into the at least one albumin dropwise between the dissolving and the mixing.

In some embodiments, the ratio of the at least one diester derivative of β-lapachone and the at least one albumin ranges from about 1:1 to about 1:20.

In some embodiments, the homogenizing was performed at about 1300 bar. In some embodiments, the homogenizing was performed at 0° C.

As used herein, the term “administer” or “administering” refers to introduce by any means a compound or composition (e.g., a therapeutic agent) into the body of a mammal in order to prevent or treat a disease or condition (e.g., cancer).

As used herein, the term “cancer” refers to a proliferative disorder disease caused or characterized by the proliferation of cells, which have lost susceptibility to normal growth control. The term “cancer,” as used in the present disclosure, includes tumors and any other proliferative disorders. Cancers of the same tissue type originate in the same tissue, and may be divided into different subtypes based on their biological characteristics. The cancer may be selected, for example, from glioblastoma, squamous cell carcinoma, skin cancer-related tumors, breast cancer, head and neck cancer, gynecological cancer, urinary and male genital cancer, bladder cancer, prostate cancer, bone cancer, endocrine adenocarcinoma, digestive tract cancer, major digestive/organ cancer, central nervous system cancer, and lung cancer.

As used herein, the terms “treating,” “treatment,” “therapy,” and “therapeutic treatment” as used herein refer to curative therapy, prophylactic therapy, or preventative therapy. These terms also describe the management and care of a mammal for the purpose of combating a disease, or related condition, and include the administration of a composition to alleviate the symptoms, side effects, or other complications of the disease or condition. Therapeutic treatment for cancer includes, for example, surgery, chemotherapy, radiation therapy, gene therapy, and immunotherapy.

“Pharmaceutically effective amount” encompasses an amount sufficient to ameliorate or prevent a symptom or sign of the medical condition. A pharmaceutically effective amount also means an amount sufficient to allow or facilitate diagnosis. An effective amount for a particular patient or veterinary subject may vary depending on factors such as the condition being treated, the overall health of the patient, the method route and dose of administration and the severity of side effects. A pharmaceutically effective amount can be the maximal dose or dosing protocol that avoids significant side effects or toxic effects. The effect will result in an improvement of a diagnostic measure or parameter by at least 5%, such as by at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter shown by a normal subject. A pharmaceutically effective amount of albumin nanoparticle would be an amount that is, for example, sufficient to reduce a tumor volume, inhibit tumor growth, or prevent or reduce metastasis.

The term “pharmaceutically acceptable” refers to those compounds, materials, compositions, and/or dosage forms, which are suitable for use in contact with the tissues of human beings and animals without excessive toxicity, irritation, allergic response, or other problem or complication, commensurate with a reasonable benefit/risk ratio.

The pharmaceutical compositions disclosed herein can also contain other conventional pharmaceutically acceptable ingredients, generally referred to as carriers, diluents, or auxiliaries, as necessary or desired. Conventional procedures for preparing such compositions in appropriate dosage forms can be utilized. Such ingredients and procedures include those described in the following references: Powell, M. F. et al, “Compendium of Excipients for Parenteral Formulations”, PDA Journal of Pharmaceutical Science ft Technology 52(5), 238-311 (1998); Strickley, R. G “Parenteral Formulations of Small Molecule Therapeutics Marketed in the United States (1999)-Part-1” PDA Journal of Pharmaceutical Science & Technology 53(6), 324-349 (1999); and Nema, S. et al, “Excipients and Their Use in Injectable Products” PDA Journal of Pharmaceutical Science Et Technology, 51 (4), 166-171 (1997).

Commonly used pharmaceutical ingredients that can be used as appropriate to formulate the composition disclosed herein for its intended route of administration include, for example, acidifying agents (examples include, but are not limited to, acetic acid, citric acid, fumaric acid, hydrochloric acid, and nitric acid); alkalinizing agents (examples include, but are not limited to, ammonia solution, ammonium carbonate, diethanolamine, monoethanolamine, potassium hydroxide, sodium borate, sodium carbonate, sodium hydroxide, and triethanolamine, trolamine); adsorbents (examples include, but are not limited to, powdered cellulose and activated charcoal); aerosol propellants (examples include, but are not limited to, carbon dioxide, chlorofluorocarbon such as Freon-11 (CCl₃F), Freon-13 (CCIF3) and Freon-114 (mostly CCIF₂—CClF₂)); air displacement agents (examples include, but are not limited to, nitrogen and argon); antifungal preservatives (examples include, but are not limited to, benzoic acid, butylparaben, ethylparaben, methylparaben, propylparaben, and sodium benzoate); antimicrobial preservatives (examples include, but are not limited to, benzalkonium chloride, benzethonium chloride, benzyl alcohol, cetylpyridinium chloride, chlorobutanol, phenol, phenylethyl alcohol, phenylmercuric nitrate and thimerosal); antioxidants (examples include, but are not limited to, ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, and sodium metabisulfite); binding materials (examples include, but are not limited to, block polymers, natural and synthetic rubber, polyacrylates, polyurethanes, silicones, polysiloxanes and styrene-butadiene copolymers); buffering agents (examples include, but are not limited to, potassium metaphosphate, dipotassium phosphate, sodium acetate, sodium citrate anhydrous, and sodium citrate dihydrate); carrying agents (examples include, but are not limited to, acacia syrup, aromatic syrup, aromatic elixir, cherry syrup, cocoa syrup, orange syrup, syrup, corn oil, mineral oil, peanut oil, sesame oil, bacteriostatic sodium chloride for injection, and bacteriostatic water for injection); chelating agents (examples include, but are not limited to, edetate disodium and edetic acid); colorants (examples include, but are not limited to, FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (examples include, but are not limited to, bentonite); emulsifying agents (examples include, but are not limited to, acacia, cetomacrogol, cetyl alcohol, glyceryl monostearate, lecithin, sorbitan monooleate, and polyoxyethylene 50 monostearate); encapsulating agents (examples include, but are not limited to, gelatin and cellulose acetate phthalate); flavorants (examples include, but are not limited to, anise oil, cinnamon oil, cocoa, menthol, orange oil, peppermint oil, and vanillin); humectants (examples include, but are not limited to, glycerol, propylene glycol, and sorbitol); levigating agents (examples include, but are not limited to, mineral oil and glycerin); oils (examples include, but are not limited to, arachis oil, mineral oil, olive oil, peanut oil, sesame oil, and vegetable oil); ointment bases (examples include, but are not limited to, lanolin, hydrophilic ointment, polyethylene glycol ointment, petrolatum, hydrophilic petrolatum, white ointment, yellow ointment, and rose water ointment); penetration enhancers for, for example, transdermal delivery (examples include, but are not limited to, monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and urea); plasticizers (examples include, but are not limited to, diethyl phthalate and glycerol); solvents (examples include, but are not limited to, ethanol, corn oil, cottonseed oil, glycerol, isopropanol, mineral oil, oleic acid, peanut oil, purified water, and sterile water for injection; stiffening agents (examples include, but are not limited to, cetyl alcohol, cetyl esters wax, microcrystalline wax, paraffin, stearyl alcohol, white wax, and yellow wax); suppository bases (examples include, but are not limited to, cocoa butter and polyethylene glycols); surfactants (examples include, but are not limited to, benzalkonium chloride, nonoxynol 10, oxtoxynol 9, polysorbate 80, sodium lauryl sulfate, and sorbitan mono-palmitate); suspending agents (examples include, but are not limited to, agar, bentonite, carbomers, carboxymethylcellulose sodium, hydroxyethyl cellulose, hydroxypropyl cellulose, hydroxypropyl methylcellulose, kaolin, methylcellulose, tragacanth, and veegum); sweetening agents (examples include, but are not limited to, aspartame, dextrose, glycerol, mannitol, propylene glycol, saccharin sodium, sorbitol, and sucrose); tablet anti-adherents (examples include, but are not limited to, magnesium stearate and talc); tablet binders (examples include, but are not limited to, acacia, alginic acid, carboxymethylcellulose sodium, compressible sugar, ethylcellulose, gelatin, liquid glucose, methylcellulose, non-crosslinked polyvinyl pyrrolidone, and pregelatinized starch); tablet and capsule diluents (examples include, but are not limited to, dibasic calcium phosphate, kaolin, lactose, mannitol, microcrystalline cellulose, powdered cellulose, precipitated calcium carbonate, sodium carbonate, sodium phosphate, sorbitol, and starch); tablet coating agents (examples include, but are not limited to, liquid glucose, hydroxyethyl cellulose, hydroxypropyl cellulose, hydroxypropyl methylcellulose, methylcellulose, ethylcellulose, cellulose acetate phthalate, and shellac); tablet direct compression excipients (examples include, but are not limited to, dibasic calcium phosphate); tablet disintegrants (examples include, but are not limited to, alginic acid, carboxymethylcellulose calcium, microcrystalline cellulose, polacrillin potassium, cross-linked polyvinylpyrrolidone, sodium alginate, sodium starch glycollate, and starch); tablet glidants (examples include, but are not limited to, colloidal silica, corn starch, and talc); tablet lubricants (examples include, but are not limited to, calcium stearate, magnesium stearate, mineral oil, stearic acid, and zinc stearate); tablet/capsule opaquants (examples include, but are not limited to, titanium dioxide); tablet polishing agents (examples include, but are not limited to, carnuba wax and white wax); thickening agents (examples include, but are not limited to, beeswax, cetyl alcohol and paraffin); tonicity agents (examples include, but are not limited to, dextrose and sodium chloride); viscosity increasing agents (examples include, but are not limited to, alginic acid, bentonite, carbomers, carboxymethylcellulose sodium, methylcellulose, polyvinyl pyrrolidone, sodium alginate, and tragacanth); and wetting agents (examples include, but are not limited to, heptadecaethylene oxycetanol, lecithins, sorbitol monooleate, polyoxyethylene sorbitol monooleate, and polyoxyethylene stearate).

The pharmaceutical composition disclosed herein can be dehydrated by at least one method selected, for example, from lyophilization, spray-drying, fluidized-bed drying, wet granulation, and other suitable methods known in the art. When the composition is prepared in solid form, such as by wet granulation, fluidized-bed drying, and other methods known to those skilled in the art, the albumin is, for example, applied to the pharmaceutically active agent, and other excipients if present, as a solution.

There are a wide variety of suitable formulations of the pharmaceutical composition disclosed herein (see, e.g., U.S. Pat. No. 5,916,596). The following formulations and methods are merely exemplary and are in no way limiting. Formulations suitable for oral administration include, for example, (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient. Such excipients are known in the art.

Formulations suitable for parenteral administration include, for example, aqueous and non-aqueous, isotonic sterile injection solutions, which can contain anti-oxidants, buffers, bacteriostats, and/or solutes that render the formulation isotonic with the blood of the intended recipient, and aqueous and non-aqueous sterile suspensions that can include suspending agents, solubilizers, thickening agents, stabilizers, and/or preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind as set forth above. In some embodiments, the injectable formulations are used.

Formulations suitable for aerosol administration comprising the pharmaceutical composition disclosed herein include, for example, aqueous and non-aqueous, isotonic sterile solutions, which can contain anti-oxidants, buffers, bacteriostats, and/or solutes, as well as aqueous and non-aqueous sterile suspensions that can include suspending agents, solubilizers, thickening agents, stabilizers, and/or preservatives, alone or in combination with other suitable components, which can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

Other suitable formulations include, for example, suppositories that can be prepared by use of a variety of bases such as emulsifying bases or water-soluble bases. Formulations suitable for vaginal administration can, for example, be presented as pessaries, tampons, creams, gels, pastes, foams, or spray formulas containing, in addition to the active ingredient, at least one carrier that is known in the art to be appropriate.

The dose of the pharmaceutical composition disclosed herein administered to a human varies with, for example, the type of the particular pharmaceutical composition, the method of administration, and the particular site being treated. The dose should be sufficient to affect a desirable response, such as a therapeutic or prophylactic response against a particular disease, within a desirable time frame.

While any suitable means of administering the pharmaceutical composition disclosed herein to the human can be used, the pharmaceutical composition disclosed herein is, for example, administered to the human via intravenous administration, intra-arterial administration, intrapulmonary administration, oral administration, inhalation, intravesicular administration, intramuscular administration, intra-tracheal administration, subcutaneous administration, intraocular administration, intrathecal administration, or transdermal administration.

Anti-cancer drugs used herein include, for example, alkylating agents such as cyclophosphamide, ifosfamide, busulfan, melphalan, bendamustine hydrochloride, nimustine hydrochloride, ranimustine, dacarbazine, procarbazine hydrochloride, and temozolomide; antimetabolites such as methotrexate, pemetrexed sodium, fluorouracil, doxifluridine, capecitabine, tegafur, cytarabine, cytarabine ocfosfate hydrate, enocitabine, gemcitabine hydrochloride, mercaptopurine hydrate, fludarabine phosphate, nelarabine, pentostatin, cladribine, levofolinate calcium, calcium folinate, hydroxycarbamide, L-asparaginase, and azacitidine; antitumor antibiotics such as doxorubicin hydrochloride, daunorubicin hydrochloride, pirarubicin, epirubicin hydrochloride, idarubicin hydrochloride, aclarubicin hydrochloride, amrubicin hydrochloride, mitoxantrone hydrochloride, mitomycin C, actinomycin D, bleomycin, peplomycin sulfate, and zinostatin stimalamer; microtubule inhibitors such as vincristine sulfate, vinblastine sulfate, vindesine sulfate, vinorelbine tartrate, paclitaxel, docetaxel hydrate, and eribulin mesylate; hormonal agents such as anastrozole, exemestane, letrozole, tamoxifen citrate, toremifene citrate, fulvestrant, flutamide, bicalutamide, medroxyprogesterone acetate, estramustine phosphate sodium hydrate, and leuprolide acetate; platinum drugs such as cisplatin, miriplatin hydrate, carboplatin, nedaplatin, and oxaliplasin; topoisomerase I inhibitors such as irinotecan hydrochloride hydrate and nogitecan hydrochloride; topoisomerase II inhibitors such as etoposide and sobuzoxane; cytokines such as interferon γ1a, teceleukin, and celmoleukin; antibody drugs such as trastusumab, rituximab, gemtuzumab ozogamicine, bevacizumab, and cetuximab; radioimmunotherapeutic agents such as ibritumomab tiuxetan; molecular target drugs such as gefitinib, imatinib mesylate, bortezomib, erlotinib hydrochloride, sorafenib tosylate, sunitinib malate, thalidomide, nilotinib hydrochloride hydrate, dasatinib hydrate, lapatinib tosylate hydrate, everolimus, lenalidomide hydrate, dexamethasone, temsirolimus, vorinostat, tretinoin, and tamibarotene; non-specific immune stimulants such as OK-432, dry BCG, Coriolus versicolor polysaccharide formulation, lentinan, and ubenimex. Other examples of the anti-cancer agents include aceglatone, porfimer sodium, talaporfin sodium, ethanol, and arsenic trioxide.

Examples of the anti-cancer drugs include anthracycline anti-cancer agents such as doxorubicin hydrochloride, daunorubicin hydrochloride, pirarubicin, epirubicin hydrochloride, idarubicin hydrochloride, aclarubicin hydrochloride, amrubicin hydrochloride, and mitoxantrone hydrochloride; platinum anti-cancer agents such as cisplatin, miriplatin hydrate, carboplatin, nedaplatin, and oxaliplatin; and pyrimidine antimetabolite-based anti-cancer agents such as fluorouracil, doxifluridine, capecitabine, tegafur, cytarabine, cytarabine ocfostate hydrate, enocitabine, and gemcitabine. In some embodiments, the anti-cancer drug is gemcitabine.

Gemcitabine is a nucleoside analogue chemotherapeutic agent that exerts its anti-tumor effect by inhibiting deoxyribonucleic acid (DNA) synthesis. In some embodiments, the form of gemcitabine is provided as the pharmaceutically acceptable hydrochloride salt of gemcitabine. The compound and methods of making and using this compound, including for the treatment of cancer, such as for the treatment of leukemias, sarcomas, carcinomas and myelomas, are disclosed in U.S. Pat. No. 5,464,826. Alternative names for gemcitabine as the pharmaceutically acceptable hydrochloride salt include Gemzar®, CAS number 122111-03-9, LY188011 hydrochloride and cytidine, 2′-deoxy-2′,2′- difluoro-, hydrochloride (1:1).

As used herein, a “PARP Inhibitor” refers to a compound whose primary activity is the inhibition of PARP activity, including PARP1 and PARP2. PARP inhibitors, include but not limit to, rucaparib, olaparib, niraparib, veliparib, iniparib, BMN-673, niraparib, talazoparib, pamiparib, iniparib, fluazolepali, simmiparib and 3-aminobenzamide.

Exemplary non-limiting combinations and uses of the anti-PD-1 antibody molecules are disclosed in U.S. Patent Appl. Pub. No. 2015/0210769 (U.S. application Ser. No. 14/604,415), entitled “Antibody Molecules to PD-1 and Uses Thereof,” incorporated by reference in its entirety. In some embodiments, anti-PD-1 antibody may be selected from Nivolumab, Pembrolizumab, Pidilizumab, and AMP514 (Amplimmune).

Exemplary non-limiting combinations and uses of the anti-PD-L1 antibody molecules are disclosed in U.S. Patent Appl. Pub. No. 2016/0108123 (U.S. application Ser. No. 14/881,888), entitled “Antibody Molecules to PD-L1 and Uses Thereof,” incorporated by reference in its entirety. In some embodiments, the anti-PD-L1 anbibody may be selected from YW243.55.S70, MPDL3280A, MEDI-4736, MSB-0010718C, and MDX-1105.

As used herein, “radiotherapy,” also called “radiation therapy,” refers to the treatment of cancer and other diseases with ionizing radiation. Ionizing radiation deposits energy that injures or destroys cells in the area being treated (i.e., the “target tissue”) by damaging their genetic material, making it impossible for these cells to continue to grow. Although radiation damages both cancer and normal cells, the normal cells are able to repair themselves and function properly. Radiotherapy may be used to treat localized solid tumors, such as cancers of the skin, tongue, larynx, brain, breast, or uterine cervix. It can also be used to treat leukemia and lymphoma (cancers of the blood-forming cells and lymphatic system, respectively). Exemplary radiotherapy may be selected from the forms of electromagnetic waves, such as X-rays or gamma rays, or charged particles or neutral particles. The radiotherapy may be administered by external beam, an interstitial implant, or a combination thereof.

As used herein, the term “chemotherapy” refers to the use of chemical agents to destroy cancer cells. Exemplary chemotherapy agents include, but are not limited to, actinomycin D, adriamycin, altretamine, asparaginase, bleomycin, busulphan, capecitabine, carboplatin, carmustine, chlorambucil, cisplatin, CPT-11, cyclophosphamide, cytarabine, dacarbazine, daunorubicin, doxorubicin, epirubicin, etoposide, fludarabine, fluorouracil, gemcitabine, hydroxyurea, idarubicin, fosfamide, irinotecan, liposomal doxorubicin, lomustine, melphalan, mercaptopurine, methotrexate, mitomycin, mitozantrone, oxaliplatin, procarbazine, steroids, streptozocin, taxol, taxotere, taxotere, temozolomide, thioguanine, thiotepa, tomudex, topotecan, treosulfan, UFT (Uracil-Tegufur), vinblastine, vincristine, vindesine, and vinorelbine. Chemotherapy may be used alone or in combination to treat some types of cancers. Sometimes it can be used together with other types of treatment such as surgery, radiotherapy, immunotherapy, or a combination thereof. In some embodiments, the albumin nanoparticle is administered as a pharmaceutical composition. In some embodiments, the albumin nanoparticle is present in a pharmaceutically effective amount.

In some embodiments, the albumin nanoparticles bind to K-Ras(G12C) without any effect on the K-Ras wild type. In some embodiments, the albumin nanoparticles selectively bind to K-Ras(G12C) without any effect on the K-Ras wild type.

In some embodiments, the cancer is associated with K-Ras wild-type or mutations. In some embodiments, the cancer is associated with K-Ras(G12C).

In some embodiments, the cancer is chosen from breast cancer, lung cancer, pancreatic cancer, colorectal cancer, gall bladder cancer, thyroid cancer, bile duct cancer, ovarian cancer, endometrial cancer, prostate cancer, and esophageal cancer. In some embodiments, the cancer is breast cancer. In some embodiments, the cancer is lung cancer. In some embodiments, the cancer is pancreatic cancer. In some embodiments, the cancer is colorectal cancer. In some embodiments, the cancer is gall bladder cancer. In some embodiments, the cancer is thyroid cancer. In some embodiments, the cancer is bile duct cancer. In some embodiments, the cancer is ovarian cancer. In some embodiments, the cancer is endometrial cancer. In some embodiments, the cancer is prostate cancer. In some embodiments, the cancer is esophageal cancer.

In some embodiments, the present disclosure provides methods for modulating an activity of a K-Ras protein, comprising contacting a K-Ras protein with an effective amount of the albumin nanoparticle. In one embodiment, the K-Ras protein is K-Ras(G12C).

In some embodiments, the present disclosure provides for methods for inhibiting KRas G12C activity in a cell, comprising contacting the cell in which inhibition of KRas G12C activity is desired with an effective amount of an albumin nanoparticle or pharmaceutical compositions containing the albumin nanoparticle. In one embodiment, the contacting is in vitro. In one embodiment, the contacting is in vivo.

In one embodiment, an albumin nanoparticle is administered in combination with at least one additional anti-cancer drug and/or at least one anti-cancer therapy.

In a further embodiment, the at least one additional anti-cancer drug is selected from gemcitabine, a PARP inhibitor, and an immunotherapeutic agent.

In one embodiment, the at least one additional anti-cancer drug is an anti-PD-1 antibody. In one embodiment, the at least one additional anti-cancer drug is an anti-PD-L1 antibody.

In another embodiment, the at least one anti-cancer therapy is selected from radiotherapy, chemotherapy, and immunotherapy.

Also provided herein is an albumin nanoparticle or a pharmaceutical composition thereof as defined herein for use in therapy.

Also provided herein is an albumin nanoparticle or a pharmaceutical composition thereof as defined herein for use in the treatment of cancer.

Also provided herein is the use of an albumin nanoparticle or a pharmaceutical composition thereof as defined herein in the manufacture of a medicament for the treatment of cancer.

Also provided herein is the use of an albumin nanoparticle, as defined herein, in the manufacture of a medicament for the treatment of a cancer associated with K-Ras wild-type or mutations.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The following examples further illustrate the present disclosure but, of course, should not be construed as in any way limiting its scope.

### EXAMPLES

### Materials

β-lap and dC₃ are synthesized by BioDuro (Beijing). BSA (bovine serum albumin) was purchased from Amresco, USA. PEG_{5K}-b-PLA_{5K}, porcine liver esterase (PLE), dicoumarol (DIC) and 5-[N-ethyl-N-isopropyl] amiloride (EIPA) were purchased from Sigma USA and used in accordance with the guidance. Organic solvents (analytical grade), hydroxypropyl-β-cyclodextrin (HP-β-CD), β-CD), phosphate buffered saline solution (PBS, pH 7.4), DAPI and fluorescein isothiocyanate (FITC)-BSA were purchased from Thermo Fisher Scientific, USA. 6-TAMRA-NHS ester (6-Carboxytetramethylrhodamine N-succinimidyl ester) was purchased from MedChem Express (New Jersey, USA). 6-TAMRA-BSA was synthesized according to common methods in the laboratory of the inventors. SW1990 and SW1990/GEM cell lines were provided by Hanmei Pharmaceutical Co., Ltd. (Beijing). Other cell lines were purchased from American Type Culture Collection (ATCC) and cultured according to ATCC instructions. All cell lines were reconfirmed by short tandem repeat (STR) DNA analysis. All animal experiments were conducted in accordance with the Guideline of Laboratory Animal Use and Care Committee of Tsinghua University.

Synthesis of 6-TAMRA-BSA comprised mixing fluorescent substance 6-carboxytetramethylrhodamine (6-TAMRA) with bovine serum albumin in an excess molar ratio of 3:1 in a solution. Then the mixture was stirred at room temperature for 2-4 hours in the dark. HiTrap™ Desalting columns (GE Healthcare) were used to filter and remove excess unreacted 6-TAMRA, and then centrifugation was performed at 3500 rpm for 10 minutes for ultrafiltration concentration. The 6-TAMRA labeled albumin obtained after the reaction should be used within one week.

### Preparation of dC₃ Micelles and Albumin Nanoparticles (BSA-NPs)

dC₃ micelles were prepared by thin film hydration method. Briefly, appropriate amounts of dC₃ and PEG_{5K}-b-PLA_{5K} were dissolved in acetonitrile and evaporated under vacuum by rotary evaporator (BUCHI R100, Switzerland) in water bath at 60° C. to form a solid film. After that, saline at 60° C. was added for hydration under ultrasound for 5 minutes. The resulting drug-loaded polymer micelle aqueous solution was filtered through a 0.45 μm membrane filter to remove unloaded drug aggregates.

dC₃ albumin nanoparticles (BSA-NPs) were prepared by high-pressure homogenization. Briefly, dC₃ was dissolved in a mixture of chloroform and ethanol (v/v=9/1), added to a BSA solution dropwise, and then homogenized with a mixer (UltraTurrax IKA, T25, Germany) at 10,000 rpm for 2 minutes. The resulting suspension was further homogenized in an ice bath with a high-pressure homogenizer (ATS Engineering, AH-2010, Shanghai) at 1300 bar for 5 minutes. Then, the resulting dC₃ BSA-NP aqueous solution was lyophilized (SP SCIENTIFIC, AdVantage 2.0 BenchTop Freeze Dryer, Canada) for later use.

### Characterization of Particle Size and Morphology for dC₃ Micelles and BSA-NPs

The particle size of the micelles and BSA-NP solution was analyzed by dynamic light scattering (DLS, Malvern Instruments Inc., U.K.) with a scattering angle of 90° at 25° C. The morphology of polymer micelles and BSA-NPs were studied by transmission electron microscopy (TEM, FEI Tecnai Spirit Bio TWIN TEM D1297, USA), wherein phosphotungstic acid was used for staining. High performance liquid chromatography (HPLC, Shimadzu LC-20AT, Kyoto, Japan) was used to measure drug load and drug encapsulation efficiency. Crystallization of dC₃ was monitored by a polarizing optical microscope (POM, Zeiss Axio Imager A2m microscope, Germany).

Kinetics Study on Release of dC₃ and β-lap from dC₃-loaded micelles and BSA-NP

The dC₃ released from the micelles and BSA-NPs was determined by HPLC using a traditional dialysis bag method. The micelles and BSA-NPs containing the same amount of dC₃ were added to the dialysis bag (MW: 8000-14000, MD 25 mm, VisKase, US) and incubated at 37° C. in a 1M sodium salicylate solution with shaking at 60 rpm. A sodium salicylate solution (1M) was used to maintain sink conditions of the dC₃. At predetermined time points (5, 10, 15, 30, 45, 60, 90, and 120 minutes), each 0.5 mL of release medium were taken out and replaced with an equal volume of fresh medium. The release medium taken above was mixed with 0.5 mL of acetonitrile for analysis of dC₃ concentration by HPLC.

The kinetics of in vitro conversion of dC₃ to β-lap in micelles and BSA-NPs were measured by previously-reported procedures. The micelles and BSA-NPs containing the same amount of dC₃ were added to a 1M sodium salicylate solution containing 1 U/mL porcine liver esterase (PLE). The solution was kept at 37° C. with shaking at 60 rpm. At predetermined time points (5, 10, 15, 30, 45, 60, 90, and 120 minutes), each 0.5 mL of release medium were taken out and replaced with an equal volume of fresh culture medium. The removed release medium was mixed with 0.5 mL of acetonitrile, and then centrifuged (Thermo Scientific Sorvall Legend Micro 21R, Thermo Fisher Scientific, USA) for 10 minutes at 10,000 rpm to remove denatured proteins. The concentration of β-lap was analyzed by H PLC.

To simulate the blood environment, the kinetics of the above-mentioned in vitro conversion of dC₃-β-lap in micelles and BSA-NPs were also measured in a 40 mg/mL albumin solution. All other conditions were maintained.

Conversion Experiments of dC₃ to β-lap at Different BSA Concentrations In Vitro

In order to study the effect of BSA on the conversion of dC₃ to β-lap catalyzed by an esterase, dC₃ was dissolved in DMSO (final DMSO concentration <0.5%), and then added dropwise to a solution of 1 M sodium salicylate, 1 U/mL porcine liver esterase (PLE) and different concentrations of BSA. The solution was shaken in a water bath at 60 rpm at 37° C. At predetermined time points (5, 10, 15, 30, 45, 60, 90, and 120 minutes), each 0.5 mL of release medium were taken out and replaced with an equal volume of fresh medium. The removed release medium was mixed with 0.5 mL of acetonitrile, and centrifuged for 10 minutes at 10,000 rpm to remove denatured proteins. The concentration of β-lap was analyzed by H PLC.

In order to study whether the effect of BSA on dC₃-β-lap conversion catalyzed by an esterase will change in the presence of the polymer, the above-mentioned experiments were also conducted with the amount of PEG_{5K}-β-PLA₅K proportional to micelle preparation. Other conditions remained unchanged.

### Determination of Methemoglobin

In order to assess the risk of methemoglobin produced by dC₃ micelles, dC₃BSA-NPs and β-lap HP-β-CD preparations (β-lap-CD), three preparations with the same amount of β-lap were quickly mixed in the blood of Sprague Dawley rats (Vital River Laboratory Animal Technology, Beijing), and incubated with 1 U/mL PLE in a water bath at 37° C. At predetermined time points (5, 10, 15, 30, and 45 minutes), each 50 μL of sample was taken out and treated using a methemoglobin determination kit (Jian Cheng Bioengineering Institute, Nanjing, China). UV/Vis absorptions (at 587 nm and 624 nm, respectively) of hemoglobin (Hb) and methemoglobin (MetHb) were determined by UV/Vis spectrophotometry (Ultrospec 9000PC, GE Healthcare, England). The concentration of MetHb (Cₘ) was calculated by following equation:

$$\begin{matrix}{E}_{x} = {E}_{m} \times {C}_{m} + {E}_{h} \times {C}_{h} & ( 1 ) \\ R = \frac{{E}_{m}}{E} , r = \frac{{E}_{h}}{E} , {C}_{m} = \frac{{E}_{x} - rE}{E ( R - r )} & ( 2 )\end{matrix}$$

wherein Eₘ is the maximum absorption at 624 nm when Hb was completely oxidized to MetHb; Eₕ is the maximum absorption at 587 nm when MetHb was completely reduced to Hb; Eₓ is the absorption measured at 624 nm, and E is the absorption at isoabsorptive points of Hb and MetHb; and Cₘ and Cₙ are the concentrations of MetHb and Hb, respectively.

Maximum Tolerated Dose (MTD) and Toxicity of dC₃ Micelles, dC₃ BSA-NPs and β-lap-CD Preparations in BALB/c Nude Mice

In order to assess the safety of dC₃ micelles, dC₃ BSA-NPs and β-lap-CD, a dose escalation study was conducted to obtain the maximum tolerated dose (MTD) of the three preparations in BALB/c nude mice (Vital River Laboratory Animal Technology, Beijing). After intravenous administration of the preparations with different doses, once every three days for a total of 4 times, the morbidity and mortality responses are recorded. Animal responses were recorded immediately after injection. Changes in body weight of the animals were recorded after each injection.

Pharmacokinetics of dC₃ and β-lap after Intravenous Injection of dC₃ Micelles and BSA-NPs

The comparison of pharmacokinetics of different preparations in Sprague Dawley rats (n=3 per group) was performed. After tail vein injection of 50 mg/kg (β-lap equivalent dose) dC₃ micelles and BSA-NPs, blood samples were taken from the fundus venous plexus at predetermined time points (5 minutes, 10 minutes, 20 minutes, 30 minutes, 45 minutes, 1 hour, 2 hours, and 4 hours). The blood samples were centrifuged at 4° C. and 1000 rpm for 3 minutes. The plasma concentrations of β-lap and dC₃ were analyzed by LC-MS/MS (AB Sciex QTRAP 5500, USA).

In Vitro Cytotoxicity of Two Dosage Forms on Pancreatic Cancer Cell Lines with Different KRAS Genotypes and Expression Levels

KRAS mutant cells MIA PaCa-2 (KRAS^{G12C}) and KRAS wild-type (KRAS^{WT}) BxPC-3 were selected for experiments. The expression levels of mutant KRAS in MIA PaCa-2 and BxPC-3 cells were manipulated. According to the supplier's protocol, MIA PaCa-2 cells with low expression level of KRAS were realized by lentiviral-mediated construction and transfection of pCDH-KRas-shRNA; control MIA PaCa-2 cells were realized by lentiviral-mediated transfection of pCDH vector; BxPC-3 cells with high expression level of KRAS were realized by lentiviral-mediated construction and transfection of pCDH-KRas-G12V, and control BxPC-3 cells were realized by lentiviral-mediated transfection of pCDH vector. All plasmids were purchased from Santa Addgene (USA). The plasmid was transfected into the cells using transfection reagents (Promega, USA). 1 μg/mL puromycin was added to the culture medium for 48 hours to select stable transfected cell lines. Cells were represented as MIA PaCa-2 control, MIA PaCa-2 KRAS KD, BxPC-3 control and BxPC-3 KRAS^{G12V}.

Briefly, four pancreatic cancer cell lines were firstly inoculated into a 96-well culture plate (4000 cells/well), each well containing 0.1 mL of cell culture medium. After 24 hours, the cell culture medium was removed and replaced with 0.2 mL of cell culture medium containing β-lap, β-lap with dicoumarin (DIC, an NQO1 inhibitor), dC₃, dC₃ micelles, and BSA-NP solution. PLE (1 U/mL) was added concurrently with the dC₃ preparation. The cells were further incubated for 12 hours, then the culture medium containing drugs was removed and new culture medium without drug was added. Relative survival curves of the four pancreatic cancer cell lines were obtained using the MTS assay (Promega, USA). The mean value of 6 wells per treatment was calculated and the treatment/control (T/C) value was calculated as a percentage of cell survival rate. All calculations were performed by GraphPad Prism 7 software (GraphPad Software, USA).

The relative survival profiles of pancreatic cancer cells SW1990 (KRAS^{G12D}) and SW1990/GEM (KRAS^{G12D}) were also measured using the same method. SW1990/GEM was cultured in a culture medium with 50 μM gemcitabine to maintain drug resistance.

Analysis of BSA Macropinocytosis of Pancreatic Cancer Cell Lines with Different KRAS Genotypes and Expression Levels by Flow Cytometry and Multiphoton Confocal Microscopy

4 pancreatic cancer cell lines, MIA PaCa-2 control, MIA PaCa-2 KRAS KD, BxPC-3 control and BxPC-3 KRAS^{G12V} (10⁵ cells per well, 1 mL) were inoculated in a 12-well microplate (Corning, N.Y., USA) and incubated at 37° C. and 5% CO₂. When the cell density reached approximately 65%, the cell culture medium was replaced with a serum-free culture medium. After 12 hours, the culture medium was removed again and replaced with DMEM containing 1 mg/mL 6-TAMRA-BSA (500 μL/well). After incubated for another 4 hours, the cells were washed, digested and suspended in PBS (400 μL) and subjected to fluorescence analysis on a flow cytometer (BD, FACS Calibur, USA) using a 544 nm laser excitation and a 575 nm emission filter. Based on forward scattering and side scattering analysis, viable cells (10,000 cells/sample) were counted to detect the fluorescence intensity of the cells. For the EIPA (a known macropinocytosis inhibitor) inhibition experiment, cells were incubated with serum-free culture medium for 12 hours, pretreated with 75 μM of EIPA for 30 minutes, then replaced with fresh DMEM containing 1 mg/ml 6-TAMRA-BSA was added (500 μL/well).

Cells were cultured and treated as described above and grown on coverslips. After cell nuclei were stained with DAPI, the cells grown on the coverslips were observed under a multiphoton confocal Ti-E A1RMP inverted microscope (Nikon, Japan).

For SW1990 and SW1990/GEM cell lines, 1 mg/mL FITC-BSA was used, and 488 nm laser excitation and 520 nm emission filter were selected for flow cytometry.

LC-MS/MS Analysis of Drug Concentrations in Cells Treated with Different Preparations

Cells were cultured and treated on a 12-well microplate as described above. When the cell density reached approximately 80%, the cell culture medium was replaced with DMEM containing dC₃ micelles and dC₃ BSA-NP to a final concentration of 2 μM dC₃. After incubated for another 2 hours, the cells were washed and lysed. The lysates were added into acetone and centrifuged to precipitate proteins. The dC₃ and β-lap concentrations in the supernatant were analyzed by LC-MS/MS to calculate the total drug content in the cells.

The EIPA inhibition experiment comprises pretreating cells with 75 μM of EIPA for 30 minutes before adding different preparations containing DMEM (500 μL/well each).

Anti-Tumor Effects of Different Preparations on Pancreatic Cancer Cell Xenograft Mouse Models with Different KRAS Genotypes and Expression Levels

MIA PaCa-2 control cells (1.0×10⁷), MIA PaCa-2 KRAS KD cells (1.0×10⁷), BxPC-3 control cells (1.0×10⁷) and BxPC-3 KRAS^{G12V} cells (1.0×10⁷) were respectively injected subcutaneously into the right groin of 6-8 week-old BALB/c nude mice to establish four cells-derived xenograft (CDX) models. When the tumor volume reached about 200 mm³, each group of CDX mice was divided into 5 groups (n>6), and then treated with saline (control), β-lap-CD (20 mg/kg), and dC₃ micelles (50 mg/kg equivalent dose of β-lap) or dC₃ albumin nanoparticles (50 and 100 mg/kg equivalent dose of β-lap). The preparations were administered via a tail vein, once every three days for 4 repetitions. During the experiment, tumor volume was calculated as (length×width×width)/2. After the tumor volume reached a certain level, the mice were sacrificed and the tumors were removed and weighed for comparison.

The long-term surviving study comprises injecting MIA PaCa-2 cells (1.0×10⁶) and BxPC-3 cells (1.0×10⁶) in situ into the pancreas of 6-8-week-old BALB/c nude mice to establish an in-situ model. The administration method and interval are the same as above. Cancer-related deaths or body weight loss of more than 20% relative to the beginning of treatment were all expressed as deaths. And animal mortality was recorded over time.

In addition, a xenograft mouse model derived from SW1990/GEM (5.0×10⁶) cells is established to verify the anti-tumor effects of different preparations. All other conditions are unchanged.

Drug Accumulation of Micelles and BSA-NPs in Pancreatic Cancer Tumor Tissues with Different KRAS Genotypes and Expression Levels

MIA PaCa-2 control cells (1.0×10⁷), MIA PaCa-2 KRAS KD cells (1.0×10⁷), BxPC-3 control cells (1.0×10⁷) and BxPC-3 KRAS^{G12V} cells (1.0×10⁷) were respectively injected subcutaneously into the right groin of 6-8 week-old BALB/c nude mice to establish four cells-derived xenograft (CDX) models. When the tumor volume reached about 400 mm³, each group of CDX mice was divided into 2 groups (n=4), and then treated with dC₃ micelles and BSA-NPs (50 mg/kg equivalent dose of β-lap) via a tail vein. After 2 hours, the mice were sacrificed, and the tumors were removed and triturated with acetonitrile. After centrifugation, the dC₃ and β-lap concentrations in the supernatant were analyzed by LC-MS/MS to calculate the total internalized drug content.

The EIPA inhibition experiment comprises pretreating mice by intratumoral injection of 5 μg of EIPA for 30 minutes before administration of different preparations via a tail vein.

### Results

The Strong Binding between dC₃-BSA Slows the Drug Release from BSA-NPs than from Micelles

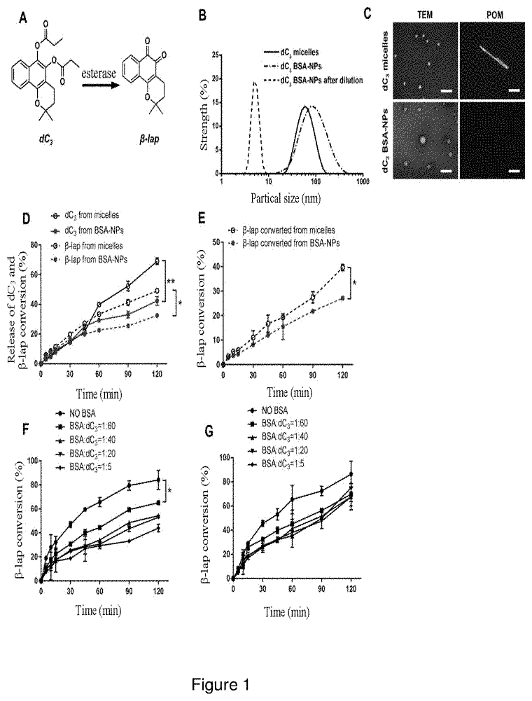

The dC₃ micelles and BSA-NPs were prepared. The theoretical dC₃ loading of 10 wt %, and the actual drug loading measured by experiments are 9.87±0.47 and 10.03±0.41 wt %, respectively. Almost all drugs were loaded (>98%) in both PEG-b-PLA micelles and BSA-NPs preparations. By dynamic light scattering (DLS), the dC₃ BSA-NPs have an average particle size of 110 nm and a polydispersity index (PdI) value of 0.21, whereas the dC₃ micelles have an average particle size of 55 nm, and a PdI of 0.12 (FIG. 1B).

TEM showed both dC₃ micelles and BSA-NPs were spherical (FIG. 1C). Although the solubility of dC₃ is less than 0.1 μg/mL, both preparations achieved an apparent drug concentration higher than 5 mg/mL (β-lap equivalent dose), which is necessary for intravenous administration. Both preparations were maintained a physically-stable state at 4° C. overnight. However, neither of the preparations can maintain the original particle size distribution when diluted in PBS. A 500-fold dilution was performed to reach 10 μg/mL (β-lap equivalent dose), dissociation of dC₃ micelles was observed by a polarizing light microscope (POM) and solid dC₃ crystals were observed to precipitate from the solution. However, even a 1000-fold dilution was performed, no solid dC₃ precipitation was observed in BSA-NP (FIG. 1C). In contrast, BSA-NPs with the original particle size of about 110 nm were dissociated into particles with a particle size of 5-10 nm after 1000-fold dilution according to DLS (FIG. 1B), wherein the dC₃-BSA complex may be composed of a single albumin molecule or a small amount of albumin molecules, indicating the presence of strong dC₃-BSA specific or non-specific binding to allow the complex of dC₃ with BSA.

Then a two-hour in vitro dissolution study was performed to compare dC₃ release and β-lap conversion of BSA-NP and micelle preparations. Without catalyzing with esterase, dC₃ was released from the micelles much faster than from BSA-NP (FIG. 1D, solid line). At 2 hours, about 69% of dC₃ were released from the micelles, while only about 42% of dC₃ were released from BSA-NP. When PLE (1 U/mL) was introduced into the release medium, dC₃ micelles produce β-lap (dotted line, FIG. 1D) faster than BSA-NPs. After 2 hours, about 49% of dC₃ from micelles were converted to β-lap, whereas about 32% of dC₃ from BSA-NPs were converted to β-lap.

In order to simulate the blood environment, β-lap conversion experiments of the two preparations were performed simultaneously in a 4% albumin solution (FIG. 1E). Similarly, the conversion of BSA-NPs prodrugs into β-lap was slower compared to micelles. Even compared to the absence of albumin, the presence of large amounts of albumin delayed the conversion of dC₃ to β-lap in both preparations.

The effect of albumin on enzymatic dC₃-β-lap conversion in vitro was investigated. As the ratio of BSA:dC3 in solution increases, it was anticipated that the balance of dC₃-BSA binding benefited to the formation of dC₃-BSA complexes, thereby delaying the dC₃-β-lap conversion catalyzed by PLE. Even when BSA was introduced at a low BSA-dC₃ ratio, the dC₃-β-lap conversion ratio was significantly reduced (FIG. 1F). In addition, when a small amount of PEG-b-PLA was added proportional to the micelle preparation, dC₃-β-lap conversion was accelerated (FIG. 1G). It seems that PEG-b-PLA interrupted the protection of BSA on the conversion of dC₃ to β-lap.

In summary, dC₃ is tightly bound to BSA, causing a much slower release of dC₃ from BSA-NPs than from micelles. The presence of albumin also significantly delays the enzymatic dC₃-β-lap conversion, and BSA production will be partially interfered by the polymers for micelle preparation. Therefore, the main differences between dC₃ micelles and BSA-NPs lie in: the dC₃ is rapidly released from the micelles and is subsequently converted to β-lap; whereas the dC₃ is slowly released from BSA-NP and a smaller amount of β-lap are produced, because most dC₃ still binds to albumin to maintain the complex state to avoid enzymatic conversion. This complex still maintains its advantages even in a 4% albumin solution, indicating that the process of conversion of dC₃ to β-lap in micelles cannot be delayed as that in BSA-NPs even in a blood circulation. This may be caused by the presence of polymers which interferes with the production of albumin-dC₃ complexes. These characteristics of the preparations may lead to significant differences in systemic toxicity (methemoglobinemia) and the preparations have drug delivery selectivity to PDAC with different expression levels of mutant KRAS, which are discussed in detail in the following sections.

Albumin Nanoparticles may Reduce Methemoglobinemia Caused by β-lap and Improve Safety

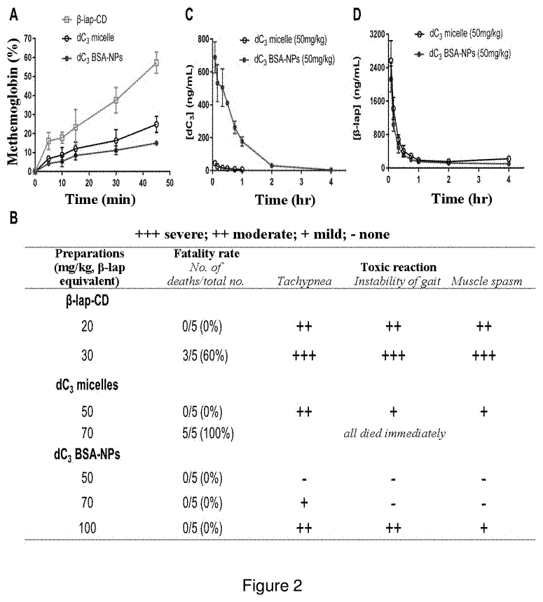

The rate of methemoglobin production in the case of dC₃ micelles and BSA-NPs were measured. β-Lap-CD was used as a positive control. As shown in FIG. 2A, compared to dC₃ micelles and β-lap-CD, dC₃ BSA-NP has a slower β-lap conversion, resulting in lower levels of methemoglobin produced in blood. Safety comparisons of β-lap-CD, dC₃ micelles, and dC₃ BSA-NP in healthy BALB/c nude mice show different toxic reactions (FIG. 2B). For βl -lap-CD, a dose of only 20 mg/kg causes severe muscle contractions, dyspnea, and irregular gait in mice, all caused by methemoglobinemia, which is consistent with earlier reports. The micelle preparation increases the MTD to 50 mg/kg (β-lap equivalent). When the dose is further increased to 70 mg/kg, all animals were observed a sudden death. In contrast, when BSA-NPs were administered at a dose of 100 mg/kg (β-lap equivalent), only slight respiratory changes were observed; MTD increases by 5 folds compared to administration of β-lap-CD, and 2 folds compared to administration of micelle formulations; no animal died, and the animals recovered quickly within 24 hours. Therefore, the MTD of BSA-NPs is determined to be 100 mg/kg. Then the changes in body weight of healthy BALB/c nude mice were monitored after administered three different preparations (once every three days, for a total of 4 times) according to MTD of each preparation. No significant change in body weight (data not shown) was observed, which is consistent with the characteristic of β-lap having chronic safety.

Pharmacokinetic curves of dC₃ (FIG. 2C) and β-lap (FIG. 2D) in blood obtained from Sprague Dawley rats intravenously injected with dC₃ micelles and BSA-NPs at a dose of 50 mg/kg (β-lap equivalent) were compared. As shown in FIG. 2C, after the injection of dC₃ micelles, because of the rapid dC₃-β-lap conversion, the plasma dC₃ concentration rapidly dropped below the detection limit (2 ng/mL) after 1 hour, and the rats after the injection showed irregular gait associated with methemoglobinemia; when injected with BSA-NPs, the animals showed no signs of side effects and a higher (about 10-fold) dC₃ blood concentration was maintained within 4 hours. At the same time, there is no significant difference observed in terms of β-lap pharmacokinetics between the two preparations (FIG. 2D), even the area under the curve (AUC) of time-concentration of the β-lap in the micelle group is 1.5-fold that in BSA-NPs group. However, at each individual time point, plasma concentrations of BSA-NPs and β-lap in the micelle group were higher than those in the BSA-NPs group. This may be due to the following two reasons:

First, the enzymatic conversion of dC₃-β-lap occurs rapidly, wherein the biggest difference in β-lap plasma concentration occurs within the first 10 minutes after intravenous injection of the two preparations, which does not occur in the sampling pharmacokinetic study (the first sampling pharmacokinetic study was performed at 5 minutes). We believe this is a valid conclusion, because animals receiving a micelle injection clearly showed signs of methemoglobinemia immediately (<5 minutes) after administration, while animals receiving BSA-NPs did not show the signs. Second, once β-lap is converted from dC₃, the pharmacokinetic half-life thereof is too short (about 24 minutes), so the plasma concentration of β-lap measured at >5 minutes after injection drops too quickly, indicating the insensitivity to the type of preparation.

It was concluded on the basis of AUC₀₋₄ hours values, that the vast majority (more than 95%) of the drug exists in the form of β-lap rather than dC₃ after injection of the micelle preparation, and two-thirds of the drug after injection of BSA-NP exist in the form of dC₃ due to strong dC₃-albumin binding. This observation is also consistent with the in vitro determination of methemoglobin (FIG. 2A).

In summary, the advantageous safety of dC₃ BSA-NP than PEG-b-PLA micelles are due to the following reasons: 1) compared with micelles (about 55 nm), BSA-NP has a larger average diameter (about 110 nm) and a lower surface area/volume ratio, which may lead to slower release of dC₃; 2) dC₃ tightly binds to albumin to form a dC₃-BSA complex, which can significantly hinder the dC₃-β-lap conversion; 3) polymers that form micelles in the blood circulation may disrupt the production of albumin-dC₃ complexes and the protective effects thereof on dC₃-β-lap conversion. These reasons cause dC₃ to be released from BSA-NPs much more slowly, and more importantly, compared with PEG-b-PLA micelles, after the injection of BSA-NPs into blood, the in vivo conversion of dC₃ to β-lap is slow. The burst release of β-lap from PEG-b-PLA micelles after intravenous injection is the primary cause of methemoglobinemia, because β-lap oxidizes iron in hemoglobin to produce methemoglobin, which cannot transport oxygen. There is only 2% methemoglobin in the blood of healthy people, and excess methemoglobin can cause tachycardia, coma, and even death.

### KRAS Mutation Sensitizes PDAC Cells to dC₃ Albumin Preparation In Vitro

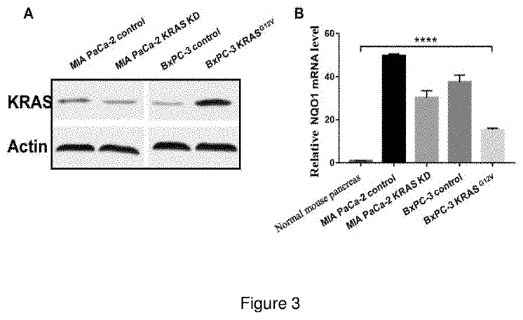

KRAS mutant cells MIA PaCa-2 (KRAS^{G12C}) and KRAS wild-type cells BxPC-3 (KRAS^{WT}) were selected for experiments to study the effects of KRAS mutations on in vitro anti-tumor effects of different preparations. The expression levels of mutant KRAS in both cell lines and transfected empty vectors for control were manipulated. The transfected cell lines were represented as MIA PaCa-2 control, MIA PaCa-2 KRAS KD, BxPC-3 control and BxPC-3 KRAS^{G12V}. The expression levels of the mutant KRAS were confirmed by Western blotting (FIG. 3A).

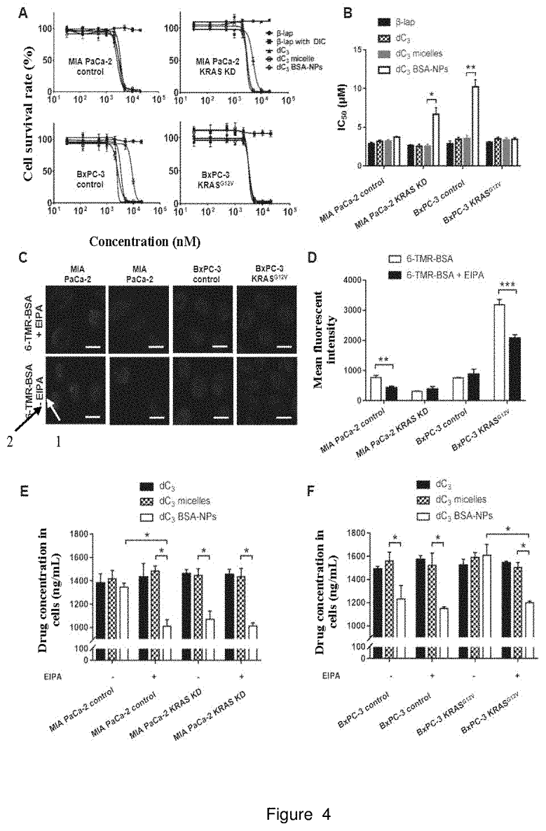

The cytotoxicity of different preparations to four human PDAC cell lines were compared, as shown in FIG. 4A. Regardless of the KRAS mutation status of the cells, all treatments were effective in inhibiting the growth of the four PDAC cells, except in the presence of DIC (a known NQO1 inhibitor), which confirmed the biological activity of β-lap NQO1. The mRNA levels of NQO1 in four human PDAC cell lines (listed in FIG. 3B) were analyzed, all four cell lines showing comparable overexpression levels of NOQ1, which is consistent with other reports.

A closer comparison of cytotoxicity among different treatments (FIG. 4B) indicated that IC₅₀ values in the KRAS mutant MIA PaCa-2 control cell line were comparable with dC₃ solutions, dC₃ micelles and dC₃ BSA-NPs, and are slightly higher than that of β-lap, which may be caused by the dC₃-β-lap conversion. Interestingly, when MIA PaCa-2 KRAS KD cells were treated, BSA-NPs showed nearly twice the IC₅₀ value compared to dC₃ solutions and micelles. The BxPC-3 (KRAS^{WT}) control had the same trend as the BxPC-3 KRAS^{G12V} . The IC₅₀ of dC₃ BSA-NPs in the BxPC-3 control instead of BxPC-3 KRAS^{G12V} cell lines was significantly higher than that of dC₃ solutions and micelles.

It is reported that mutant KRAS induces the metabolism of conversion, in which extracellular proteins such as albumin are taken up by cancer cells via enhanced macropinocytosis and lysosomal activity and provide amino acids for the cancer cells. Therefore, the degree of albumin uptake by various PDAC cells in the present disclosure, with or without inhibiting the macropinocytosis (FIGS. 4C and 4D) were compared. In fact, compared to the MIA PaCa-2 KRAS KD cell line, KRAS mutant MIA PaCa-2 control cells apparently can take up more 6-TAMRA-BSA. Moreover, BxPC-3 KRAS^{G12V} cells also internalize more 6-TAMRA-BSA than BxPC-3 (KRAS^{WT}) control cells, which can be observed and analyzed by multiphoton confocal microscopy (FIG. 4C) and flow cytometry (FIG. 4D). In addition, most of the 6-TAMRA-BSA uptake by these KRAS mutant cells was achieved via macropinocytosis, which can be inhibited by the known macropinocytosis inhibitor EIPA. In contrast, EIPA almost had no effect on BSA uptake by cells with low mutant KRAS expression levels (such as MIA PaCa-2 KRAS KD and BxPC-3 control cells), indicating that other mechanisms of endocytosis such as phagocytosis and clathrin- or caveolae-mediated endocytosis, instead of macropinocytosis, are dominant in these cell lines.

The intracellular dC₃ concentrations of the four cell lines treated with dC₃ solution, micelles, and BSA-NPs (FIGS. 4E and 4F) were further compared. Considering that the enzyme in the cells may cause the conversion of the prodrug to β-lap, the concentrations of dC₃ and β-lap were measured and the total internalized drug concentration was calculated. In the absence of EIPA, MIA PaCa-2 control cells treated with different dC₃ preparations had almost the same internalized drug concentrations. However, when EIPA was introduced, only the intake of BSA-NP was reduced. MIA PaCa-2 KRAS KD cells with or without EIPA always have a lower intake of BSA-NPs than cells treated otherwise (FIG. 4E). Internalized dC₃ was also reduced when EIPA was used to inhibit the macropinocytosis before treatment of BxPC-3 KRAS^{G12V} with BSA-NP, whereas no change occurred in KRAS^{WT} BxPC-3 control cells (FIG. 4F).

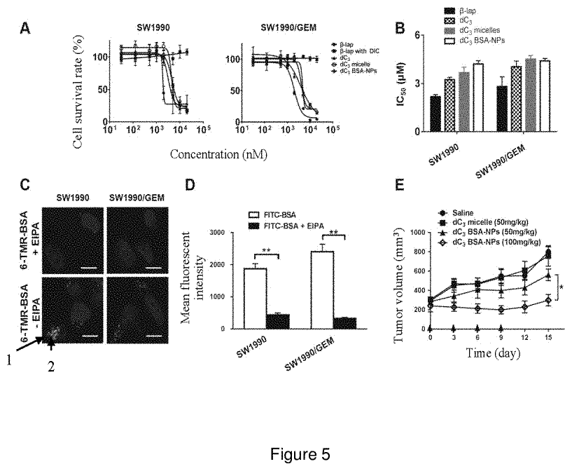

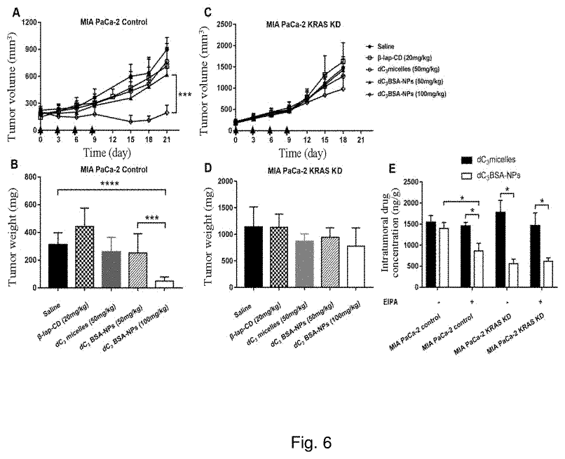

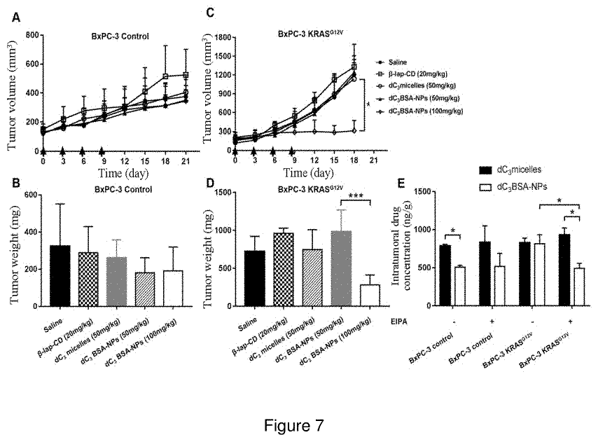

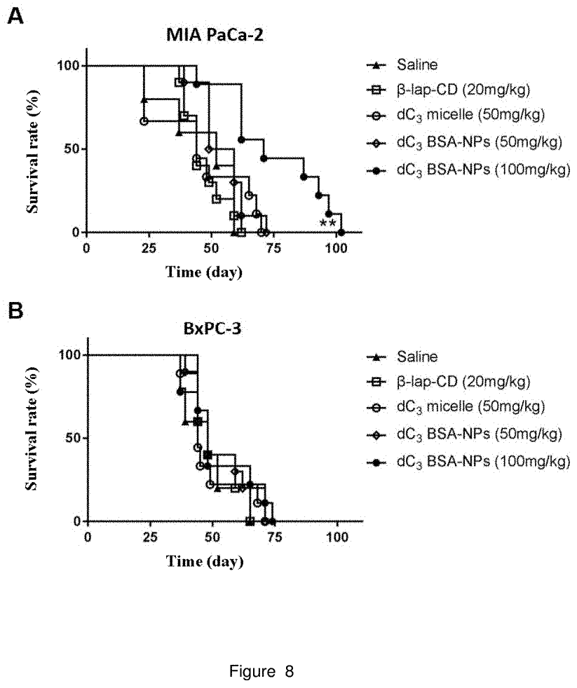

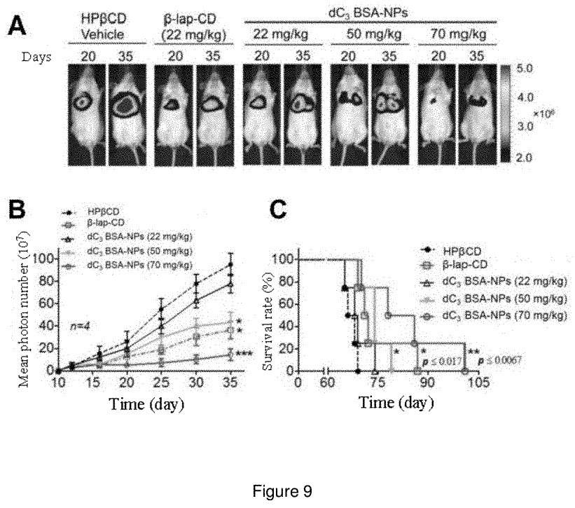

It is also noted that KRAS-enhanced macropinocytosis appears to be independent of the acquired GEM resistance, as demonstrated by comparing IC50 values and BSA uptake of SW1990 and SW1990/GEM cells of KRAS^{G12D} (FIGS. 5A to 5D). Combining the fact that β-lap can also effectively inhibit these two cells, it is concluded that dC₃ BSA-NPs provide a new strategy for KRAS mutant PDAC regardless of GEM resistance.

In summary, the above-mentioned results confirmed the KRAS mutation-dependent sensitivity of PDAC cells to BSA-NPs. As stated above, BSA-NPs were decomposed into tightly-bound albumin-dC₃ complexes with little free dC3 or β-lap. The albumin-dC₃ complex cannot be passively diffused into cells, but must be internalized by cells via phagocytosis, including macropinocytosis, which is KRAS-dependent. When treatment at an administered dose was performed, dC₃ micelles are dissociated, which is the reason why the IC₅₀ and internalized performance thereof are similar to those of treatment with dC₃ solution. Macropinocytosis is a unique mode of drug delivery to cancer cells, leading to different cytotoxicities of different dosage forms to KRAS mutant and KRAS^{WT} PDAC cells.

KRAS-Enhanced Macropinocytosis Increases PDAC Subcutaneous Transplantation Tumor Sensitivity to dC₃-BSA

In order to verify the KRAS-dependent sensitivity of PDAC to different dC₃ preparations in vivo, the anti-tumor effects of the preparations in mice carrying the above-mentioned KRAS^{G12C} MIA PaCa-2 control and MIA PaCa-2 KRAS KD cell line were compared. As shown in FIGS. 6A and 6B, for the MIA PaCa-2 control CDX model, the MTD doses of 20 mg/kg for β-lap-CD and 50 mg/kg for dC₃ micelles both failed to inhibit tumor growth. At the same 50 mg/kg dose, the BSA-NPs group also failed to inhibit tumor growth. However, due to the much higher MTD, dC₃ BSA-NPs at 100 mg/kg significantly inhibited the MIA PaCa-2 tumor growth, as seen by tumor volume (FIG. 6A) and body weight measurement (FIG. 6B) at the end of the experiment. Limited by MTD, dC3 micelles could not reach a high dose of 100 mg/kg and failed to show a therapeutic window in this animal model. For the MIA PaCa-2 KRAS KD CDX model, no preparation, even a high MTD dose of BSA-NP, ever showed significant anti-tumor effects (FIGS. 6C and 6D).

Intratumoral drug concentrations in the two cell lines treated with dC₃ micelles and BSA-NPs at the same dose of 50 mg/kg (β-lap equivalent) were further compared (FIG. 6E). Considering that the precursor may be converted into β-lap caused by enzymes in circulation and tissues, the concentration of dC₃ and β-lap by LC-MS/MS were measured and expressed as β-lap. In the absence of EIPA, MIA PaCa-2 control CDX models treated with the two preparations had almost the same intratumoral drug concentrations. However, when EIPA was introduced, only the BSA-NPs group had a reduced intratumoral drug concentration. For MIA PaCa-2 KRAS KD cells with or without EIPA, treatment with BSA-NPs always led to a lower intratumoral drug concentration than treatment with micelles (FIG. 6E).

Similar results can be obtained from a comparison between BxPC-3 control and BxPC-3 KRAS^{G12V} CDX model. A high MTD dose (100 mg/kg) of BSA-NP only had significant anti-tumor effects on BxPC-3 KRAS^{G12V} CDX model, but not on the wild-type model (FIGS. 7A to 7D). In addition, the presence of EIPA can significantly reduce the intratumoral drug concentration of BSA-NPs, but will not reduce the intratumoral drug concentration of micelles in BxPC-3 KRAS^{G12V} CDX tumors (FIG. 7E).

In fact, even at a dose of 100 mg/kg (the maximum tolerated dose), BSA-NP cannot inhibit tumors with low expression of mutant KRAS, such as the MIA PaCa-2 KRAS KD and BxPC-3 control models. It is speculated that from the results of the intratumoral drug concentration, dC₃-albumin complex cannot effectively inhibit tumor growth by entering into tumor cells due to the lack of KRAS-enhanced macropinocytosis.

dC₃-BSA Complex has Significant Advantages in Prolonging Life in the Case of KRAS-mutated PDAC Orthotopic Transplantation Tumors

In order to compare the anti-tumor effects of different preparations, long-term survival assessments of different MTDs on pancreatic orthotopic models derived from KRAS^{G12C} MIA PaCa-2 and KRAS^{WT} BxPC-3 cell lines were performed. And all data of Kaplan-Meier were plotted (FIGS. 8A and 8B). For the MIA PaCa-2 derived orthotopic tumor model, the data showed that 50% of animals in the control group treated with saline died within 52 days, and β-lap-CD and dC₃ micelles failed to show any significant increase in survival advantage when mice were treated with same at their MTD, wherein 50% of animals had the survival time of 44 days (FIG. 8A). The same dose of dC₃ BSA-NPs at 50 mg/kg also showed no effect. At a higher MTD of 100 mg/kg, the average survival time of 50% of animals was 71 days. The Kaplan-Meier survival curves showed that dC₃ BSA-NPs at 100 mg/kg had a statistically significant survival advantage over saline controls β-lap-CD or dC₃ micelles (p<0.01). In contrast, even dC₃ BSA-NPs at 100 mg/kg had no significant anti-tumor effects on the KRAS^{WT} BxPC-3 orthotopic tumor model (FIG. 8B). The KRAS-dependent efficacy of the dC₃ albumin preparation was effectively validated.

dC₃-BSA Complex can Inhibit KRAS Mutant PDAC Xenografts Resistant to Gemcitabine

In addition, the efficacy of two dC₃ preparations on inhibiting tumors in another KRAS^{G12D} PDAC in vivo model, i.e., a gemcitabine-resistant SW1990/GEM subcutaneous xenograft model (FIG. 5E), were evaluated. Interestingly, dC₃ BSA-NPs can significantly inhibit the growth of SW1990/GEM tumors not only at a dose of 100 mg/kg but also at a dose of 50 mg/kg, while the same dose of dC₃ micelles show no effect on inhibiting tumors. This result again proved that when delivered as a dC₃-BSA complex, β-lap was effective in inhibiting KRAS mutant PDAC regardless of whether the cancer has GEM resistance or not. This demonstrated the clinical value of dC₃ BSA-NP as an active drug for NQO1, which was because GEM resistance is widely observed in patients with PDAC.

KRAS mutations are widely present in different types of tumors, not only in pancreatic cancer, and therefore, the efficacy of different preparations on inhibiting tumors using the KRAS^{G12S} A549 lung cancer cell orthotopic model (FIGS. 9A to 9C) were evaluated. Bioluminescence imaging (FIG. 9A), tumor volume curve (FIG. 9B), and Kaplan-Meier survival curve (FIG. 9C) showed that dC₃ BSA-NPs at 50 and 70 mg/kg had significant anti-tumor effects, and dC₃ BSA-NPs at 70 mg/kg showed a higher advantages on inhibiting tumors than β-lap-CD at MTD dose (22 mg/kg).

In general, the results from different animal tumor models indicated that dC₃ BSA-NPs could have a higher MTD and had a higher advantage on inhibiting tumors for KRAS mutant cancers than β-lap-CD and dC₃ micelles, regardless of the gemcitabine resistance. And this inhibition was not limited to PDAC. It is believed that this albumin-based β-lap prodrug preparation provides a valuable therapeutic strategy for various KRAS mutant cancers.

What is claimed is:

1. An albumin nanoparticle, comprising:
a core consisting of at least one diester derivative of β-lapachone having a formula of (I): 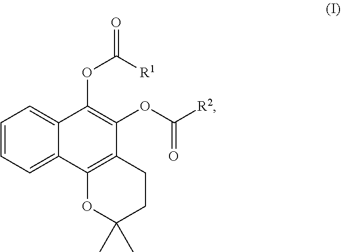
wherein
R¹ and R² are each independently selected from C₁-C₁₀ alkyl, C₂-C₆ alkenyl, C₂-C₆alkynyl, C₃-C₈ cycloalkyl, C₆-C₁₀ aryl, C₃-C₈ heterocycloalkyl, C₆-C10 heteroaryl, each of which is unsubstituted or substituted with 1 or 2 R′;
or R¹ and R² are each independently selected from a bond, —O—, —S—, —NH—, and C₁-C₄ alkylene to form a 6-10 membered ring, wherein the ring is unsubstituted or substituted with 1 or 2 R′;
further wherein each R′ is independently selected from halogen, hydroxyl, C₁-C₃ alkyl, C₁-C₃ alkoxy, C₁-C₃ haloalkyl, and —CN;
and a shell consisting of at least one albumin,
wherein the core and the shell are connected with a non-covalent bond.

2. The albumin nanoparticle according to claim 1, wherein R is selected from methyl, ethyl, propyl, butyl, pentyl, hexyl, phenyl, and naphthyl, each of which is unsubstituted or substituted with 1 or 2 R′.

3. The albumin nanoparticle according to claim 2, wherein R is selected from methyl, ethyl, propyl, and pentyl.

4. The albumin nanoparticle according to claim 3, wherein R is ethyl.

5. The albumin nanoparticle according to claim 1, wherein the at least one albumin is selected from serum-derived albumin, bioengineered recombinant albumin, and analogs thereof.

6. The albumin nanoparticle according to claim 5, wherein the serum-derived albumin is selected from human serum albumin and bovine serum albumin.

7. The albumin nanoparticle according to claim 1, wherein the albumin nanoparticle has one or more properties selected from a mean particle size ranging from about 50 to about 500 nm, a Poly Dispersity Index (PDI) ranging from about 0.01 to about 0.50, and a molar ratio of the at least one diester derivative of β-lapachone and the at least one albumin ranging from about 50:1 to about 1:1.

8. The albumin nanoparticle according to claim 7, wherein the mean particle size is about 110 nm.

9. The albumin nanoparticle according to claim 7, wherein the PDI is about 0.21.

10. The albumin nanoparticle according to claim 7, wherein the molar ratio of the at least one diester derivative of β-lapachone and the at least one albumin is about 20:1.

11. A process of making the albumin nanoparticle according to claim 1, comprising:
mixing the at least one diester derivative of β-lapachone with the at least one albumin to obtain a mixture;
homogenizing the mixture to obtain a homogenized mixture; and
lyophilizing the homogenized mixture.

12. The process according to claim 11, further comprising dispersing the at least one diester derivative of β-lapachone in at least one solvent selected from acetone, dichloromethane, chloroform, ethyl acetate, isopropyl acetate, acetonitrile, methanol, ethanol, isopropanol and mixtures thereof, prior to the mixing.

13. The process according to claim 12, wherein the at least one solvent is a mixture of chloroform and methanol.

14. The process according to claim 12, wherein the at least one diester derivative of β-lapachone has a concentration ranging from about 50 to about 200 mg/mL after the dissolving.

15. The process according to claim 11, wherein the at least one albumin is in an aqueous solution having a concentration ranging from about 2 to about 50 mg/mL.

16. The process according to claim 12, further comprising adding the at least one diester derivative of β-lapachone into the at least one albumin dropwise between the dissolving and the mixing.

17. The process according to claim 11, wherein the molar ratio of the at least one diester derivative of β-lapachone and the at least one albumin used in the process ranges from about 1:1 to about 20:1.

18. The process according to claim 11, wherein the homogenizing is performed at about 1300 bar, 0° C.

19. An albumin nanoparticle according to claim 1, made by a process selected from de-solvation, self-assembly, emulsification, double emulsification, thermo gel, spray drying, nanoparticle albumin-bound (nab) technology, and pH agglomeration.

20. A pharmaceutical composition, comprising the albumin nanoparticle according to claim 1, and at least one pharmaceutically acceptable carrier.

21. A method of treating cancer by administering a patient in need thereof a therapeutically effective amount of the albumin nanoparticle of claim 1.

22. The method according to claim 21, wherein the cancer is associated with KRAS mutations.

23. The method according to claim 22, wherein the cancer is selected from breast cancer, lung cancer, pancreatic cancer, colon cancer, rectal cancer, gall bladder cancer, thyroid cancer, bile duct cancer, ovarian cancer, endometrial cancer, prostate cancer, and esophageal cancer.

24. The method according to claim 23, wherein the cancer is pancreatic cancer.

25. The method according to claim 24, wherein the pancreatic cancer is pancreatic ductal adenocarcinoma.

26. The method according to claim 23, wherein the lung cancer is non-small cell lung cancer.

27. The method according to claim 21, further comprising administering at least one additional anti-cancer drug and/or at least one anti-cancer therapy.

28. The method of claim 27, wherein the at least one additional anti-cancer drug is selected from gemcitabine, a PARP inhibitor selected from olaparib, niraparib, veliparib, rucaparib, talazoparib, pamiparib, iniparib, fluazolepali, simmiparib and 3-aminobenzamide, and an immunotherapeutic agent selected from an anti-PD-1 antibody and an anti-PD-L1 antibody.

29. The method of claim 27, wherein the at least one anti-cancer therapy is selected from radiotherapy, chemotherapy, and immunotherapy.
